(19) United States
(12) Patent Application Publication
(10) Pub. No.: US 2018/0355387 A1
(43) Pub. Date: Dec. 13, 2018

(54) CELLULOSIC BIOFUEL AND CO-PRODUCTS

(71) Applicant: ICM, Inc., Colwich, KS (US)

(72) Inventors: Jeremy Edward Javers, St. Joseph, MO (US); Christopher Riley William Gerken, Helena, MO (US); Samuel Vander Griend, Wichita, KS (US); Jesse Spooner, Easton, MO (US); Jonathan Phillip Licklider, Cosby, MO (US); Charles C. Gallop, Gower, MO (US); Oreste J. Lantero, Trimble, MO (US); Brandon Emme, Kansas City, MO (US); Ryan Edward Hoefling, Faucett, MO (US)

(21) Appl. No.: 15/735,107

(22) PCT Filed: Jun. 10, 2016
(86) PCT No.: PCT/US2016/037072

§ 371 Date: May 21, 2018

Related U.S. Application Data
(60) Provisional application No. 62/173,936, filed on Jun. 11, 2015.

(51) Int. Cl. C12P 7/64; C12N 1/18; C12P 7/10

(52) U.S. Cl. CPC C12P 7/649; C12N 1/18; D21C 1/02; C12P 2201/00; C12P 7/10

(57) ABSTRACT

This disclosure describes processes for using biomass feedstock to produce a fermented product and co-products. The process includes washing the biomass feedstock, pretreating the washed feedstock, hydrolysis and fermentation of the pretreated feedstock(s) to produce cellulosic biofuel and co-products. The processes may also include yeast hydrolysis and aerobic propagation.

### CROSS REFERENCE TO RELATED APPLICATION

This application claims priority to U.S. Provisional Application No. 62/173,936 entitled “Cellulosic Biofuel and Co-Products” filed on Jun. 11, 2015, the contents of which are hereby incorporated by reference in its entirety.

### TECHNICAL FIELD

The subject matter of this disclosure pertains to treating feedstock by undergoing a variety of processes to produce cellulosic biofuel and other co-products. The processes include washing the feedstock, pretreating the feedstock, generating sugars from the feedstock, fermenting the feedstock and generating co-products from the feedstock while performing other processes in a biofuel plant that may be located adjacent to an existing plant while integrating energy, water, and nutrients between the two plants.

### BACKGROUND

The United States relies on imported petroleum to meet needs of transportation fuel. To reduce dependence on the imported petroleum, Congress passed Energy Policy Act to establish a Renewable Fuel Standard (RFS) Program. The RFS Program includes a mandate to blend renewable fuel into transportation fuel. The renewable fuel includes biomass-based diesel, advanced biofuel, and cellulosic biofuel. For 2015, the Environmental Protection Agency (EPA) proposes 16.30 billion gallons of total renewable fuel to be blended under the RFS Program. The EPA suggests that at least 10 percent of overall fuel supply used in the United States be from renewable fuel for 2016. For instance, this is an expected volume production of cellulosic biofuel at 206 million gallons. See, United States Environmental Protection Agency, Renewable Fuel Standard Program. (http://www.epa.gov/otaq/fuels/renewablefuels/documents/420f15028.pdf).

As a result of the RFS Program, new companies and/or existing ethanol plants are evaluating new technologies to produce cellulosic biofuel from a variety of feedstocks. Cellulosic biofuel is ethanol produced from lignocellulose by converting sugars in cellulose. For instance, plants are currently looking to incorporate new technologies to produce cellulosic biofuel that would be in close proximity to their existing ethanol plants, which currently converts grain starches, corn, milo, wheat, barley, sugarcane, beet, and the like to ethanol. The close proximity would provide benefits of integration of energy, nutrients and water between the existing “starch ethanol plants” and the cellulosic biofuel plants. The starch ethanol plant is used as a mere example of a plant, this process may be located adjacent to various types of plants that produce ethanol, cellulosic biofuel, or other renewable fuel products. In another embodiment, the new technologies described may be a stand-alone cellulosic biofuel plant.

The cellulosic materials are abundant as cellulose is found in plants, trees, bushes, grasses, wood, and other parts of plants (i.e., corn stover: leaves, husks, stalks, cobs). Cellulose is a component of the cell wall of green plants. However, converting cellulosic materials to cellulosic biofuel tends to be challenging.

The challenges include difficulties in releasing the sugars in the cellulosic material, inhibiting fermentation due to the by-products formed by release of the sugars produced, and the difficulties in fermenting the sugars. Another challenge includes having a process that is cost effective, as the starch ethanol plants want financial payback in a relatively short period of time. Accordingly, there are needs for converting biomass feedstock to produce cellulosic biofuel to meet the RFS mandate and to create co-products to help plants with financial payback.

### SUMMARY

This disclosure describes processes for converting biomass feedstock to produce cellulosic biofuel and co-products. This disclosure describes a method for washing feedstock, pretreating the washed feedstock by adding an acid and a base for neutralization, hydrolyzing the pretreated feedstock by adding a cellulase enzyme to produce hydrolysate, removing suspended solids from the hydrolysate to produce clarified sugars and lignin, concentrating the clarified sugars to produce concentrated sugars, and fermenting the concentrated sugars with stillage grown yeast paste to produce cellulosic biofuel.

This disclosure also describes a method for pretreating a biomass feedstock. The pretreatment method includes using evaporator condensate from concentrated sugars as water source to the biomass feedstock to create low-solids slurry, injecting sulfuric acid into the low-solids slurry after it has attained a predetermined pressure, and adding heat to the low-solids pressurized slurry.

This disclosure describes yet another method for combining a cellulosic stillage process stream and defatted stillage stream into a tank, adding a base to the tank to create a mixture, sending the mixture to be combined with a fermenting yeast in a propagation tank to create culture medium with yeast, mechanically separating the culture medium with yeast to produce yeast paste and yeast centrate. These are two co-products produced from the process.

There is yet another process that includes hydrolyzing yeast solids in a solid stream with a mixture of components, evaporating the hydrolyzed yeast to concentrated yeast, and drying the concentrated yeast to produce single cell protein.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Other aspects and advantages of the claimed subject matter will be apparent from the following Detailed Description of the embodiments and the accompanying figures.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items. The figures do not limit the claimed subject matter to specific embodiments described herein.

FIG. 1 illustrates an example overview process to produce cellulosic biofuel and multiple co-products with yeast recycle.

FIG. 2 illustrates an example overview process to produce cellulosic biofuel and multiple co-products without yeast recycle.

FIG. 3 illustrates an example process to wash biomass feedstock.

FIG. 4 illustrates an example process to pretreat biomass feedstock.

FIG. 5 illustrates an example process to add base and enzymes for enzyme hydrolysis.

FIG. 6 illustrates an example process to separate fermented materials for yeast recycle, yeast hydrolysis and aerobic propagation.

FIG. 7 illustrates an example process to separate fermented materials for yeast hydrolysis, and aerobic propagation.

FIGS. 8 and 9 illustrate example processes of yeast hydrolysis to produce cellulosic biofuel and single cell protein (SCP).

FIGS. 10 and 11 illustrate example processes of aerobic propagation to produce yeast paste and yeast centrate.

### DETAILED DESCRIPTION

Overview

This disclosure describes techniques to use biomass feedstock to produce cellulosic biofuel and multiple co-products. A benefit of producing the cellulosic biofuel includes reducing greenhouse gas emissions (GHS) by 85% over reformulated gasoline. Overall expected benefits of this disclosure include providing cost-effective cellulosic biofuel into the marketplace to reduce consumption of imported petroleum or reduce import of cellulosic ethanol as well as providing multiple co-products that add value to the cellulosic biofuel plants.

A variable that affects profitability of producing the cellulosic biofuel, include being able to co-locate these new processes next to an existing starch ethanol plant to lower the costs for commercial production of the cellulosic biofuel and producing products and co-products that are valuable to the cellulosic biofuel plants. The benefits of being located next to the existing starch ethanol plant include using existing roads, labor, water, piping, storage, energy, and loading infrastructure available at the existing starch ethanol plant. Other benefits include generating diversified products, such as heat, power, valuable animal feed, other types of co-products, and producing lignin for boiler fuel or alternative uses. In addition to these benefits, the described processes include meeting the RFS mandate by producing the cellulosic biofuel, decreasing fouling on solid surfaces that are detrimental to the function that is part of the cellulosic biofuel process, and recycling heat and power.

While aspects of described techniques can be implemented in any number of different environments, and/or configurations, implementations are described in the context of the following example environment. Although the techniques are described for a co-located process, these techniques may be applied towards building a plant separately on its own to produce the cellulosic biofuel.

Illustrative Environment

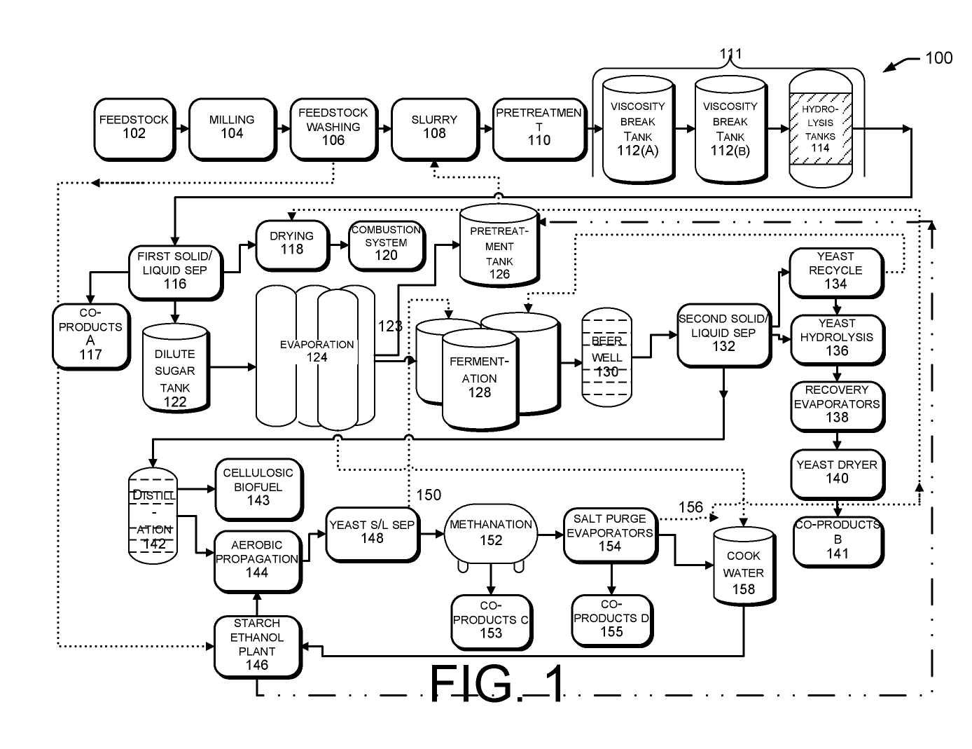

FIGS. 1-11 are flow diagrams showing example processes. The processes may be performed using different environments and equipment than what are shown in the example flow diagrams. The processes or equipment should not be construed as necessarily order dependent in their performance. Any number of the described processes or pieces of equipment may be combined in any order to implement the method, or an alternate method. Moreover, it is also possible for one or more of the provided process steps or pieces of equipment to be omitted.

FIG. 1 illustrates an example overview process 100 to produce cellulosic biofuel and multiple co-products with yeast recycle. The process 100 operates in a continuous or a batch process. The biomass feedstock may be grouped into four main categories that include, but are not limited to, (1) wood residues (including wood chips, sawmill and paper mill discards), (2) municipal waste products (including solid waste, wood waste) (3) agricultural wastes (including corn stover, corn cobs, cereal straws, hay, and sugarcane bagasse), and (4) dedicated energy crops (which are mostly composed of fast growing tall, woody grasses, including switch grass, energy/forage sorghum, and Miscanthus). The process 100 may receive biomass feedstock that includes, but is not limited to, energy sorghum, switchgrass, energy crops, other parts of plants (i.e., corn stovers: leaves, husks, stalks, cobs), Panicum virgatum, Miscanthus grass species, and the like.

The feedstock may include an individual type, a combined feedstocks of two types, or any combinations or blends of feedstocks in various percentage ranges. A cellulosic biofuel plant processes one or more biomass feedstocks to convert into cellulosic biofuel and multiple valuable co-products that include, but are not limited to, single cell protein, liquid fertilizer, lignin, methane, and ash. Other types of applications include but are not limited to, producing polymers, organic acids, chemicals, plastics, nylon, solvents, and the like.

For brevity purposes, the process of using a single feedstock will be described with reference to FIG. 1. However, the process for a combined feedstock may be similar to the process as described in FIG. 1. In an embodiment, the process 100 uses a feedstock 102 of corn stover, switchgrass, or energy sorghum with the techniques described below. The feedstock 102 is composed of cellulose, hemicellulose, and lignin. The biomass feedstock includes: the cellulose at about 30 to about 60% by weight composed of glucose, a C6 sugar; the hemicellulose is about 20 to about 40% by weight, composed of pentose/hexose/acetyl (pentose or C5 sugar) including xylose and arabinose and hexose sugar including mannose, galactose, glucose; and the lignin is about 10 to about 25% by weight, composed of aromatic alcohols. The process 100 can covert the feedstock 102 composing of the cellulose and the hemicellulose to produce cellulosic biofuel by fermentation of the simple sugars with an appropriate organism. However, the lignin component presents challenges during processing as it has a tough bonding.

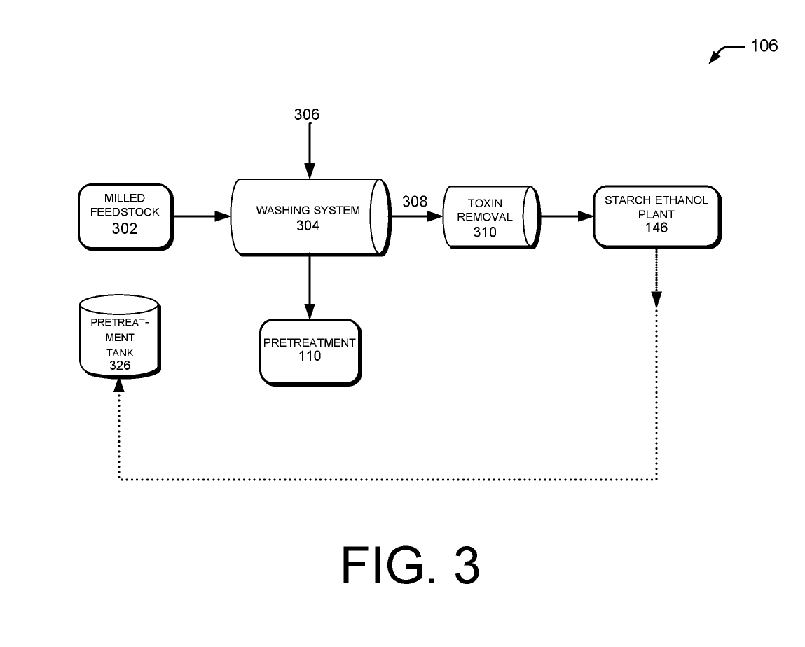

One skilled in the art understands that reducing particle size of the feedstock 102 occurs initially. At milling 104, the process 100 initially shreds the feedstock 102. The process 100 grinds the feedstock through a mechanical grinding device, such as a hammer mill, a roller mill, a knife mill, and the like. The process 100 grinds the feedstock to 50.8 millimeters or less in size (2 inches) to achieve optimal conversion during pretreatment 110 and hydrolysis 111. For instance, the process 100 reduces the feedstock to an adequate size to increase surface area-to-mass ratio for optimal exposure to contact surfaces. In an embodiment before/during/after the grinding, the process 100 removes foreign material such as rocks, sand and other foreign material by sifting, aspiration or the like. In an embodiment after the grinding, the process 100 further washes 106 the feedstock to remove toxins, dirt, soluble components, and other particles. The process 100 washes the feedstock 106, which is discussed with reference to FIG. 3. In another embodiment, the process 100 does not wash the feedstock so there is no feedstock washing, based on the conditions of the biomass feedstock (for example sugarcane bagasse would be washed in the sugar extraction process prior to entry into process 100). After feedstock washing 106, the process 100 creates a slurry 108 and sends the process stream of biomass feedstock for pretreatment 110. Condensates may be used for the slurry 108.

The use of biomass feedstock requires pretreatment 110 to open the components so enzymes may access the cellulose and the hemicellulose. The process 100 sends the feedstock 102 through pretreatment 110 to further increase its surface area, partially hydrolyzes cellulosic and hemicellulosic components, and to disrupt the lignocellulose structure for hydrolyzing agents to access cellulose component, and to reduce crystallinity of cellulose to facilitate hydrolysis.

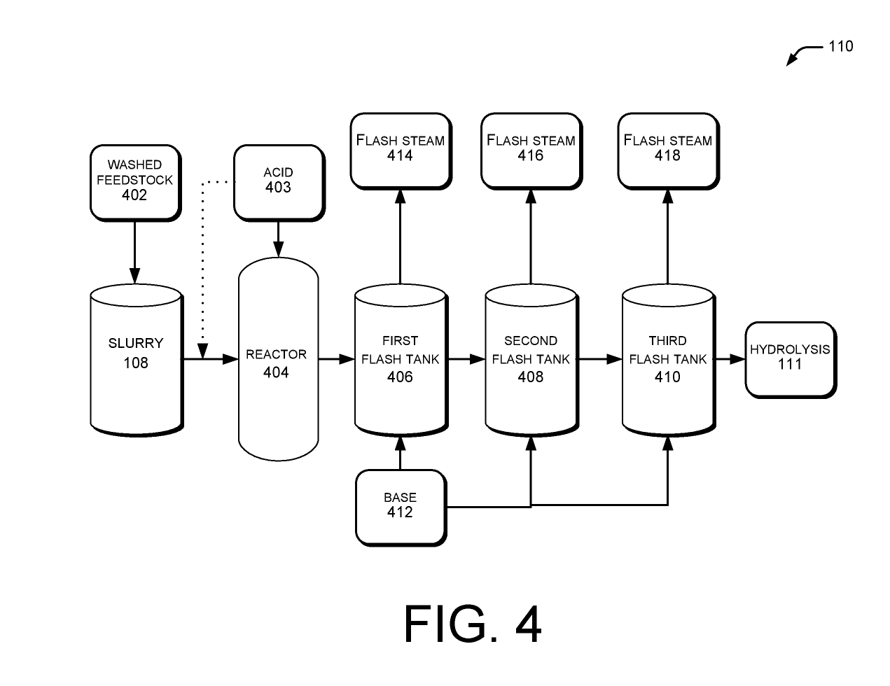

Pretreatment 110 is discussed with reference to FIG. 4. The process 100 may use pretreatment condensate generated from pretreatment 110 as cook water in the existing starch ethanol plant to maintain water balance and provide yield benefits by another 2% within the starch ethanol plant.

Next, the process 100 sends the pretreated feedstock from pretreatment 110 to hydrolysis 111, which breaks down the cellulose components to monomeric sugars. Hydrolysis 111 may include acid hydrolysis, enzymatic hydrolysis, or alkaline hydrolysis. Acid hydrolysis may include, but is not limited to, dilute acid or concentrated acid hydrolysis. Enzymatic hydrolysis breaks down the components based on the action of the enzymes. Alkaline hydrolysis breaks down the components by using a hydroxide ion. Enzymatic hydrolysis is commonly used today due to the rapid development of enzyme technologies. A person having ordinary skill in the art would be familiar with various options of hydrolysis such as dilute acid, concentrated acid, separate hydrolysis, separate hydrolysis and fermentation, simultaneous saccharification and fermentation, hybrid hydrolysis and fermentation, consolidated bioprocessing, and the like.

Hydrolysis 111 includes one or more viscosity break tank(s) 112 and one or more hydrolysis tank(s) 114 to break down the complex chains of sugars that make up the hemicellulose and the cellulose in the pretreated feedstock, occurring for about one hour to about 168 hours to reach monomeric sugar production of 80 to 99% conversion rates. Hydrolysis 111 converts the pretreated feedstock, which includes the cellulose and remaining post-pretreatment hemicellulose to glucose, soluble six-carbon sugars, mannose, galactose, xylose (i.e., soluble five-carbon sugars) and arabinose using a cellulase enzyme cocktail in a hydrolysis tank(s). The cellulase enzyme cocktail breaks down the chains of sugars of cellulose. The cellulase enzyme cocktail may include a blend of cellulase enzyme and hemicellulase enzyme (i.e., xylanase).

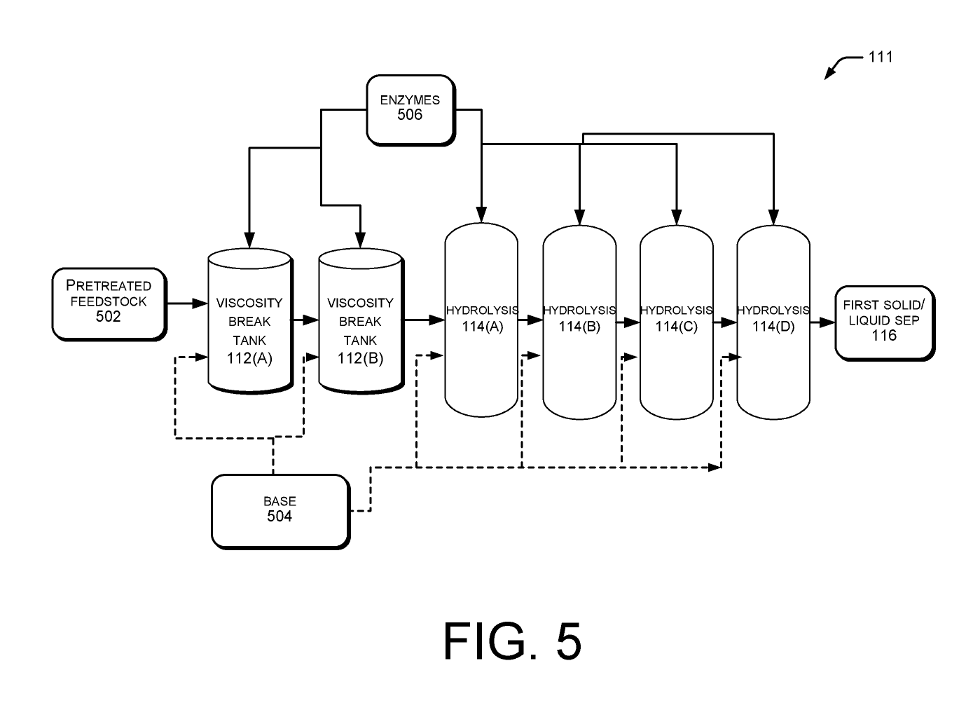

In an embodiment, hydrolysis 111 uses a cellulase and hemicellulase complex enzyme blend that degrades the cellulose and hemicellulose to fermentable sugars. It includes a blend of cellulase of advanced GH61 compounds, improved β-glucosidases, and hemicellulase. An option for use is a commercial product, Novozymes' Cellic CTec3, which is a cost-efficient solution, as less enzyme will be needed for conversion. Hydrolysis is further discussed with reference to FIG. 5.

Hydrolysis 111 may occur for about one hour to about 168 hours to achieve a target enzymatic conversion of glucan to glucose and xylan to xylose. Hydrolysis 111 lowers the temperature range of hydrolysate to about 323 K to about 328 K (about 50° C. to about 55° C., about 120° F. to about 140° F.) and the pH is controlled in a range of about 4 to about 5.5 in the hydrolysis tank(s) 114. After the process 100 provides pretreatment 110 and hydrolysis 111 to the feedstock 102, this process stream may be referred to as hydrolysate.

After hydrolysis 111, the solids tend to be present in large quantities with various particle sizes, which may make removal of the solids from the hydrolysate rather difficult. The solids may negatively affect fermentation issues and downstream processing. Mixing the hydrolysate solids with the yeast also removes the potential for generating valuable yeast SCP downstream. Thus, the process 100 uses a first solid/liquid separation 116 to separate out the solids from the hydrolysate for downstream processing. In an embodiment, the process 100 may include a heat exchanger to heat the hydrolysate to about 322 K to about 344 K (about 120° F. to about 160° F.). In an embodiment, the heat exchanger may be located after hydrolysis 111 and before the first solid/liquid separation 116.

The process 100 sends the hydrolysate through the first solid/liquid separation 116 to create unconverted solids, Co-Products A 117 (i.e., solids is a cake, which includes lignin co-products) and liquids with small particles. The first solid/liquid separation 116 may include separation equipment, including but not limited to, a centrifuge, a nozzle centrifuge, a rotary drum vacuum filter, a filter press, a leaf filter, a centrifuge with washing, an inverting filter centrifuge, a paddle screen, a multi-zoned screening apparatus, a rotary press, membrane filters, a washing stage that may be included with any of the equipment, and the like.

In an embodiment, the first solid/liquid separation 116 may include a chemical for separating, but is not limited to, chemical additives, polymers, flocculants, coagulants, inorganics, and the like. The first solid/liquid separation 116 may use the process described in U.S. patent application Ser. No. 14/586,328, entitled Separation Process filed on Dec. 30, 2014. In particular, the chemical used is GRAS approved, meaning it satisfies the requirements for the United States' Food and Drug Administration category of compounds that are “Generally Recognized As Safe.” Since the chemical is GRAS approved, it does not need to be removed and may be fed to livestock and/or other animals when used within the dosage and application guidelines established for the particular animal feed formulation. Also, the chemical may be considered a processing aid under the government agencies, such as the U.S. Food and Drug Administration, the Center for Veterinary Medicine, and the Association of American Feed Control Officials based on their standards.

For example, the process 100 may add a chemical to the hydrolysate prior to entering a single stage filter press. The single stage filter press washes the unconverted solids, which makes it amenable to co-firing in a solid fuel combustion system as well as maximizes sugar recovery for fermentation.

The first solid/liquid separation 116 removes soluble components (i.e., sugars, minerals) from the unconverted solids. The washing of the first solid/liquid separation 116 further removes nitrogen and sulfur species, which minimizes downstream mono-nitrogen oxides NO and NO₂ (i.e., NOx) and sulfur and oxygen containing compounds (i.e., SOx) emissions. The unconverted solids washing of the first solid/liquid separation 116 also increases cellulosic biofuel yield since the maximal amount of sugars are recovered from being washed. The first solid/liquid separation 116 provides a dilute clarified sugar stream of about 30 to about 90 g/L sugar glucose, xylose, arabinose, mannose, and galactose to a dilute sugar tank 122.

In an embodiment, the process 100 sends a portion of the unconverted solids to create co-products A 117 and sends a second portion of the unconverted solids through drying 118 and through a combustion system 120.

Returning to the first solid/liquid separation 116, the process 100 sends the liquids with small particles, which includes sugar-rich hydrolysate to a dilute sugar tank 122 and onto evaporation 124 to provide a concentrated sugar stream of about 150-300 g/L of sugars, including glucose, xylose, arabinose, mannose, and galactose. Evaporation 124 may include multiple effect evaporators to remove water, acetate, and furfural from the liquids with small particles.

In an embodiment, the process 100 sends the water condensed from evaporation 124 to be used as starch cook water or as pretreatment water to a pretreatment tank 126. The evaporator condensate 123 retrieved from the evaporator has acetic acid, which makes pretreatment 110 more efficient and/or improves the quality of the pretreatment 110. This provides an added benefit in integrating the processes between the starch ethanol plant and the cellulosic biofuel plant.

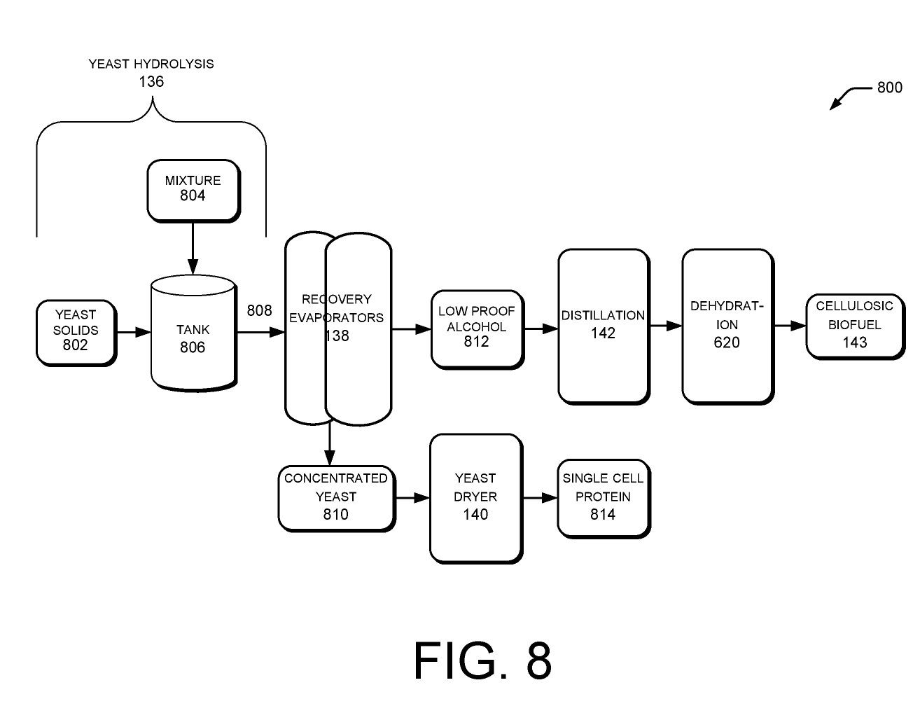

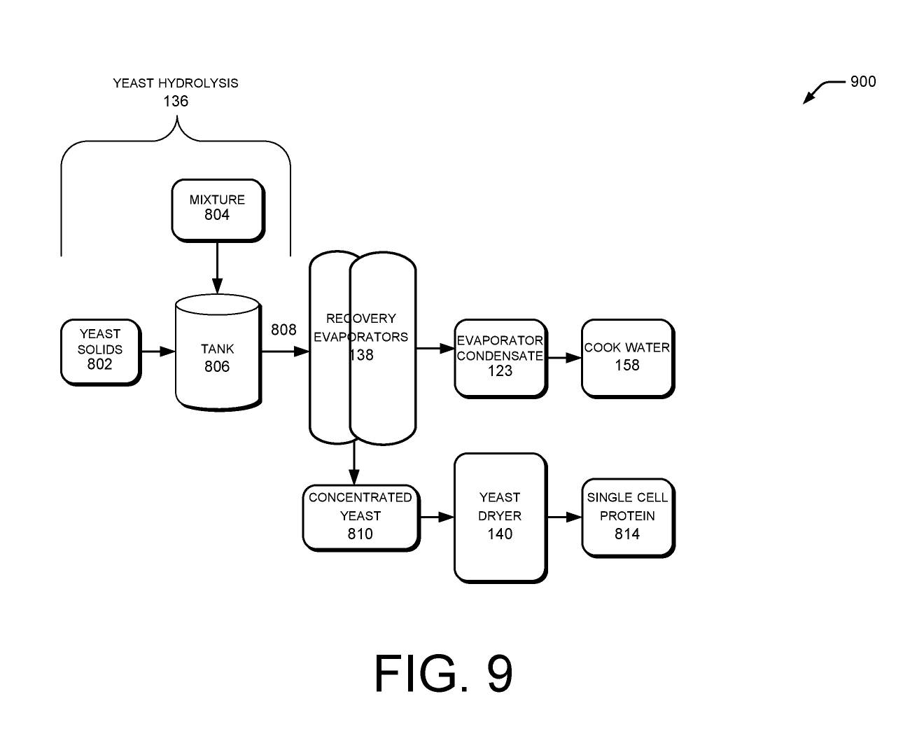

Evaporation 124 provides the concentrated sugar stream that the process 100 sends to fermentation 128 to ferment the sugars to produce cellulosic biofuel and co-products, such as single cell protein. Details of generating single cell protein, which is a valuable co-product for a plant, will be discussed with reference to FIGS. 8 and 9.

The fermentation 128 occurs in one or more fermentation tank(s) where the concentrated sugar stream is fermented to alcohol in a range of about 40 to about 90 g/L, preferably at about 6% to about 9% w/w ethanol by fed-batch fermentation. The process 100 targets a productivity level ranging from 0.3 to about 5 g alcohol per L/hour.

Fermentation 128 requires an organism(s) capable of metabolizing both 5-carbon and 6-carbon sugars present in the hydrolysate to cellulosic ethanol. A genetically modified or metabolically engineered organism may provide the most robust candidate, capable of fermenting the 6-carbon sugars typically encountered in starch ethanol processing, as well as the 5-carbon sugars resulting from the degradation of the cellulosic biomass feedstocks. For instance, fermentation 128 may convert the single sugars obtained from the hydrolysis 111 (glucose from cellulose and xylose from hemicellulose) to cellulosic biofuel. The overexpression of native traits and the addition of new traits may be desired for a yeast strain capable of efficiently utilizing the sugars present in the hydrolysate. The genetic modification of yeasts and other microorganisms is well studied and a suitable organism may be obtained from a number of suppliers who specialize in providing commercial quantities of yeast to the fuel and beverage production industries. The yeast may include, but is not limited to, a Genetically Modified Organism (GMO) yeast, a C5/C6 fermenting GMO yeast, a GMO Saccharomyces cerevisiae yeast, a Saccharomyces cerevisiae yeast, an anaerobic ethanol fermenting and aerobic glycerol consuming yeast, and such. In another embodiment, the process may use a bacteria (Escherichia coli) to convert the simple sugars to cellulosic biofuel.

The process 100 adds a yeast ranging from about 3 to about 60 g/L cell dry weight to the concentrated sugar stream in fermentation 128. In an embodiment, the process 100 adds a GMO Saccharomyces cerevisiae to ferment the C5/C6 sugars at 3 to about 60 g/L cell dry weight.

The fermentation 128 process occurs at a temperature of about 28° C. to about 36° C. (about 82.4° F. to about 97° F.), pressure ranging from about 0 psig to about 3 psig, and creating a pH level that ranges from about 4.5 to about 6.5 by adding a base. The process 100 converts the concentrated sugar stream into beer and carbon dioxide to achieve the best yield.

The fermentation 128 may be as short a process as about 20 hours or as long as about 100 hours. In embodiments, the fill rate may range from 22 to 72 hours or it may range from about 50 to about 60 hours. The process 100 places the inoculum of the GMO Saccharomyces cerevisiae into the fermentation tank(s) within 1 to 10 hours of start of fill. The residence time in the fermentation tank(s) may be about 50 to about 60 hours. In another embodiment, the process 100 may add half of the yeast required for fermentation 128 at start of fill and add the other half of the yeast required for fermentation 128 at about 30 hours. However, variables such as microorganism strain being used, rate of enzyme addition, temperature for fermentation 128, targeted alcohol concentration, size of tanks, and the like affect fermentation 128 time. The process 100 creates the alcohol, solids, and liquids in fermentation 128. Once completed, the mash is commonly referred to as beer, which may contain about 5 to 16% w/w alcohol, water, soluble and insoluble solids.

The process 100 sends the beer through a beer well 130 and through a second solid/liquid separation 132, creating three portions: a solids portion sent to yeast recycle 134, another solids portion sent to yeast hydrolysis 136 and a liquids portion sent to distillation 142. From yeast hydrolysis 136, the process 100 further sends the yeast solids through ethanol recovery evaporators 138 and yeast dryer 140 to produce Co-Products B 141, such as SCP. Yeast hydrolysis is further described with reference to FIGS. 8 and 9.

The second solid/liquid separation 132 may include separation equipment, including but not limited to, a centrifuge, a nozzle centrifuge, a rotary drum vacuum filter, a filter press, a leaf filter, a centrifuge with washing, an inverting filter centrifuge, a paddle screen, a multi-zoned screening apparatus, a rotary press, membrane filters, a washing stage that may be included with any of the equipment, and the like.

The second solid/liquid separation 132 sends the liquid portion to distillation 142, which may be carried out in two or three columns. The purpose of distillation 142 is to remove dissolved carbon dioxide from the beer and to concentrate the alcohol. Basically, the process 100 distills the beer to separate the alcohol from the solids and the liquids by going through distillation 142. Distillation 142 may include, but is not limited to, a rectifier column, a beer column, a side stripper, a beer stripper, pervaporation, or a distillation column. Any of these combinations may be used in distillation 142. The process 100 condenses the alcohol in distillation 142 and the alcohol exits through a top portion of the distillation 142 at about 90 to 95% purity, which is about 180 to 190 proof.

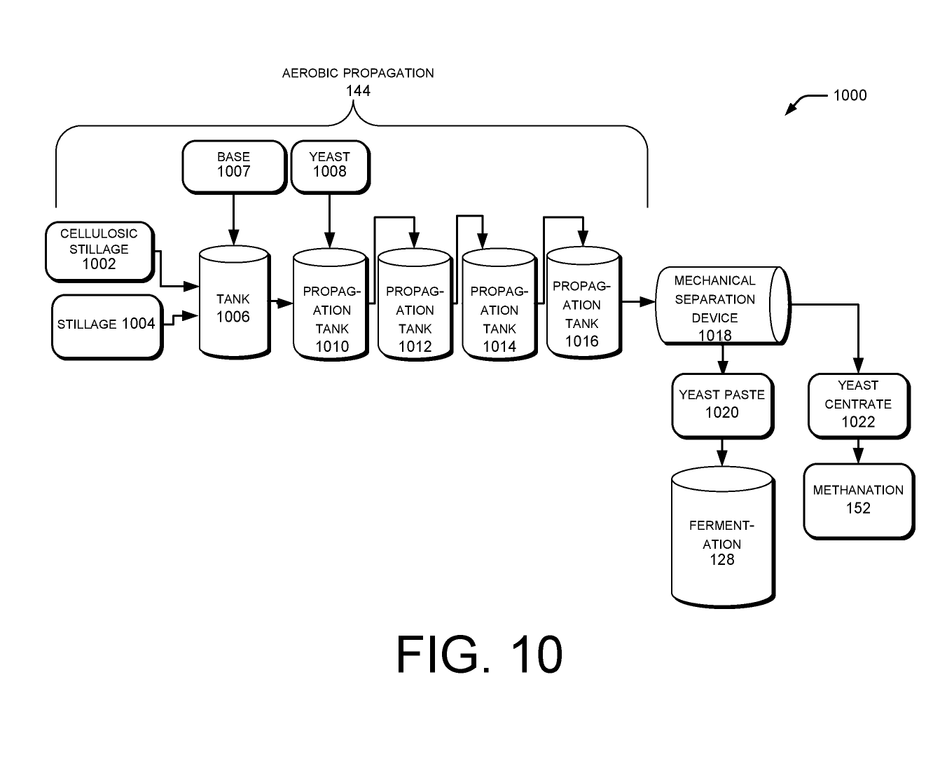

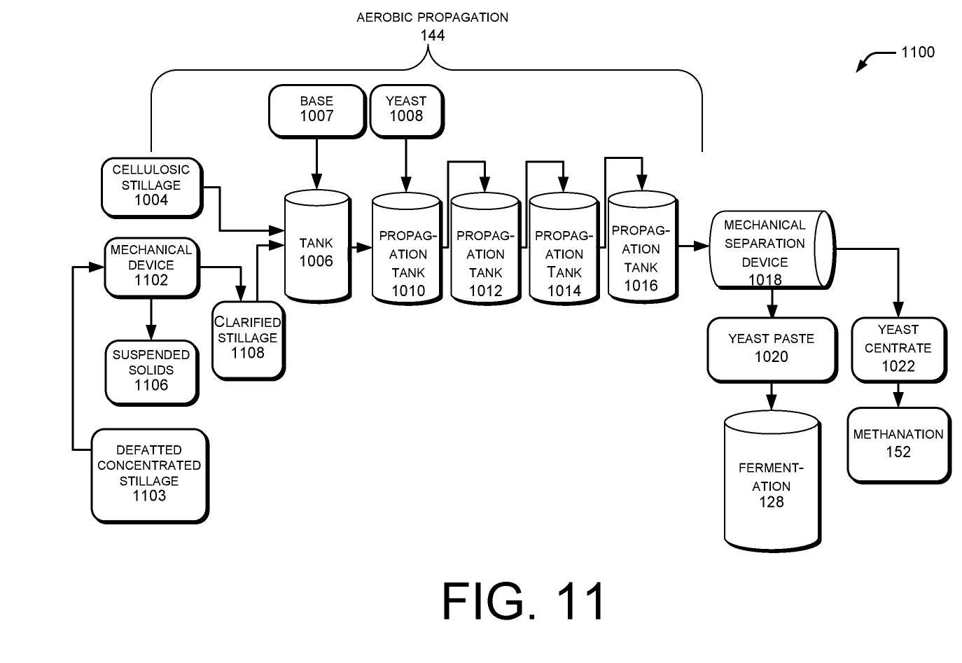

The process 100 creates valuable co-products, such as yeast paste in making SCP and yeast centrate in making methane gas, by going through a series of processes. The series of processes to create yeast paste and yeast centrate are described with reference to FIGS. 10, and 11. The process 100 may include dehydration to remove moisture from the 190 proof alcohol by going through a molecular sieve device. The dehydration includes one or more dehydration column(s) packed with molecular sieves to yield a product of nearly 100% alcohol, which is 200 proof.

The process 100 may add a denaturant to the alcohol prior to or in a holding tank. Thus, the alcohol is not meant for drinking but is to be used for motor fuel purposes. At 143, an example product that may be produced is cellulosic biofuel, to be used as fuel or fuel additive for motor fuel purposes.

In other embodiments, the process 100 may produce cellulosic biofuel after the second solid/liquid separation 132 or after distillation 142. The terms cellulosic ethanol and cellulosic biofuel are used interchangeably to describe a product produced from biomass feedstocks.

The U.S. EPA has given renewable identification number (RIN) for cellulosic biofuel as D3. The EPA uses the RIN to track biofuel trading as a unique RIN generated for each volume of biofuel produced by a plant. There is a monetary value associated with RINs as an incentive for renewable fuel production.

The process 100 further sends the process stream from distillation 142 to aerobic stillage propagation 144, which receives a process stream from the starch ethanol plant 146. From there, the process 100 sends the process stream through a yeast solid/liquid separation 148 to send a portion 150 (such as yeast slurry) to fermentation 128 and another portion (such as yeast centrate) to methanation 152, which produces Co-Products C 153, such as methane gas.

From methanation 152, the process 100 sends materials such as salt and water to one or more salt purge evaporator(s) 154 to produce Co-Products D 155, such as brine. The salt purge evaporator(s) 154 may send a stream 156 to the drying 118 and another stream to be used as cook water 158 in the starch ethanol plant 146. In another embodiment, the salt purge evaporator(s) 154 may send condensate to the pretreatment tank 126.

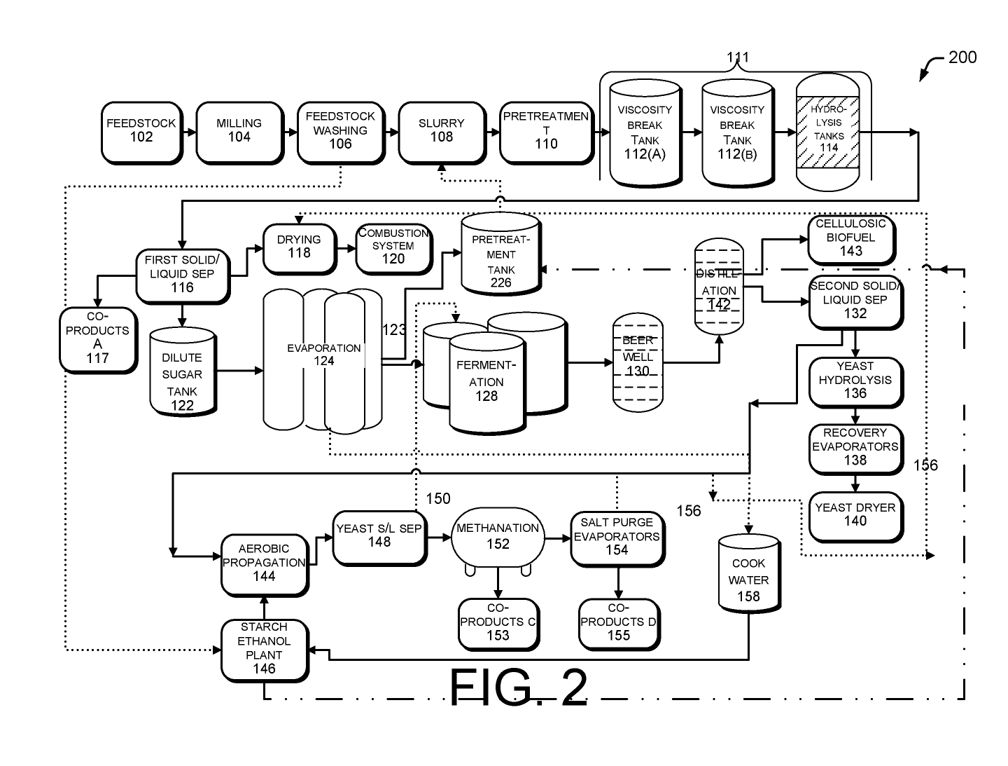

FIG. 2 is similar to the overview process of FIG. 1 with reference numbers in the 200s. It is shown as an example of the overview process 200, without yeast recycle.

Washing Feedstock

FIG. 3 illustrates a process 106 for washing the biomass feedstock 102. The process 106 receives milled feedstock 302 onto a washing system 304. The washing system 304 may include, but is not limited to, a washing table with wedge wire screen, a paddle screen, a multi-zoned screening apparatus, a counter-current washing system, a rotary press, and the like. The process 106 receives water 306, which may include clean water from the starch ethanol plant 146 that is primarily free of suspended and dissolved solids. Or in other embodiments, the water may be from process scrubber water or evaporator condensates to wash the milled feedstock 302. The temperature of the water 306 may range from about 71° C. to about 106° C. (about 160° F. to about 222° F.) or range from about 82° C. to about 100° C. (about 180° F. to about 212° F.) in different embodiments.

The process 106 washes elements, toxins, such as minerals, soluble sugars, sodium, potassium, aflatoxin, and the like, from the milled feedstock 302 in the washing system 304, and sends the washed feedstock stream 308 to pretreatment 110. Since the washing removes soluble content and/or mineral variability from the milled feedstock 302, this reduces the amount of acid needed in pretreatment 110. An amount of time for the process 106 for washing may range from about 1 minute to about 60 minutes, depending on the type of equipment and the type of feed stock.

Furthermore, the process 106 sends the used water stream 308 from the washing system 304 for toxin removal 310. Aflatoxin is a toxin, which occurs naturally as a fungi that can contaminate feedstock due to high humidity or drought conditions. Methods for toxin removal 310 from the used water stream 308 include, but are not limited to, using a chemical additive, using enzymes, adding heat, using an anaerobic digester, concentrating the toxins by chemical, physical or adsorption means, settling out the toxins, using an evaporator, and the like. Next, the process 106 sends the spent wash water in which the toxins have been removed, to the starch ethanol plant 146 as slurry make-up water and/or as cook water.

In an embodiment, the process 106 may use an aspirator to remove debris from the feedstock. The aspirator may be located prior to the washing system 304. In an embodiment, the process 106 may add a chemical additive to enhance washing performance. The chemical additive may include, but is not limited to, antifoam, wetting agent, caustic solution, caustic solution for de-acetylation, and the like. The process 106 may add the chemical additive to the milled feedstock 302 prior to or in the washing system 304.

Pretreatment of the Feedstock

FIG. 4 illustrates an example process of pretreatment 110. Pretreatment 110 may include, but is not limited to, mechanical, chemical, acid catalyzed, alkaline, biological, or combinations of physical and chemical means.

The washed feedstock 402 is composed mostly of cellulose, hemicellulose, and lignin. Cellulose and hemicellulose contain sugars that can be converted by enzymes and microorganisms to a fermented product. The use of biomass feedstock as described here requires pretreatment 110 to open the fiber so enzymes may access the cellulose and hemicellulose. However, the acid degradation of hemicellulose gives off furfural. In FIG. 4, the pretreatment 110 receives the washed feedstock 402, adds water (not shown) to wet the washed feedstock 402 in slurry 108. In another embodiment, pretreatment 110 receives milled feedstock that has not been washed. The tank used in slurry 108 may include an agitator with upflow or downflow, which agitates a low-solids slurry stream of washed feedstock 402 with the water. The pretreatment 110 may use evaporator condensate as the source of water in the slurry 108, which has a low pH. For instance, the evaporator condensate may be retrieved from evaporation 124 (i.e., first to third effect evaporators), from salt purge evaporators 154 in the cellulosic biofuel plant, or from evaporators from the starch ethanol plant 146. The condensate retrieved from the evaporator has acetic acid, which makes the pretreatment 110 more efficient and improves the quality of the pretreatment 110. The majority of the water tends to come from evaporators, distillation, or cook water.

The pretreatment 110 may add an acid 403 in line or in a reactor 404. In an embodiment, pretreatment 110 adds an acid 403 inline after a pump, to the process stream from the slurry 108. The pump (not shown) creates a pressurized zone, with the pressure being equivalent to pretreatment pressure. Adding the acid 403 after the material has entered the pressurized zone provides benefits, such as reducing the amount of high nickel alloy required in construction of tanks, which reduces capital expense.

This combination creates a low-solids slurry at about 5% to about 25% total solids. In an embodiment, the low-solids slurry ranges from about 8% to about 19% total solids. The low-solids slurry benefits the downstream processes. Thus, pretreatment 110 uses evaporator condensate as water source, creating a low-solids slurry, may add heat to the acidic low-solids slurry, agitating the acidic low-solids slurry, adjusting pH, and recycling energy.

In another embodiment, the first effect steam from the evaporator recycles a portion of pretreatment condensate directly to the slurry tank 108. In yet other embodiments, the water source for the slurry 108 comes from condensate off a flash tank and/or condensate from the ethanol starch plant 146 and/or side stripper bottoms. In another embodiment, some of the pretreatment condensate from pretreatment 110 may be recycled to the starch ethanol plant 146. It is possible to use pretreatment condensate as cook water in the starch ethanol plant 146 to decrease glycerol production. This will cause an increase in yield from the starch ethanol plant of approximately 2%. Thus, there is value in using pretreatment condensate as cook water in the starch ethanol plant.

The pretreatment 110 adds the water from pretreatment tank 126 to create the low-solids slurry in the slurry 108 to a temperature range of about 50° C. to about 100° C. (about 122° F. to about 212° F.). Options are that the water or the slurry 108 may be heated and maintained at this temperature range. The low-solids slurry has a residence time of about 1 minute to about 20 minutes in the slurry 108 with a pH of less than about 4. The residence time varies depending on the size of the slurry tank, the percent of solids, the temperature of the materials and such.

In an embodiment, the pretreatment 110 may directly inject steam to the low-solids slurry stream. The direct steam injection occurs through the heater. The heater may include one to about six heaters that may operate in a series or in parallel. Here, the heaters may add steam directly to the low-solids slurry stream past atmospheric pressure. For instance, the temperature reached is greater than about 100° C. (greater than about 212° F.). This occurs for about few seconds to about few minutes depending on the flow rate of the stream and the number of heaters being utilized in the pretreatment 110. In embodiments, there may be heating zones to heat the low-solids slurry by direct or indirect heat.

The slurry tank 108 may include a piston pump. Other embodiments include but are not limited to, a medium consistency pump, a multiple stage centrifugal pump, rotary lobe pump, progressive cavity pump, and the like. Pretreatment 110 sends the low-solids slurry stream through the piston pump to be injected with an acid 403 and then to a reactor 404.

In an embodiment, pretreatment 110 injects the acid 403 to the low-solids slurry stream to cause a reaction zone to occur in the reactor 404. This reaction zone may take about 5 minutes to about 20 minutes. This is possible due to the amount of low solids in the low-solids slurry stream. The acid 403 may include, but is not limited to sulfuric, phosphoric, and nitric acid. The concentration of the acid may typically be used at about 0.5% to about 6% w/w of the dry solids of the low-solids slurry stream. For example, in an embodiment, the pretreatment 110 uses sulfuric acid at about 2% to about 4% w/w of the dry solids of the washed feedstock 402. The pH is less than 2 for the low-solids slurry stream that has been injected with the acid 403. Thus, pretreatment 110 adjusts the pH from about 4 to less than 2 for the low-solids slurry stream. In embodiments, the acid 403 may be injected in the process stream, added at an inlet of a reactor, or at any desired point of the reactor.

The reactor 404 further hydrolyzes the cellulose and hemicellulose in the low-solids slurry. The reactor 404 has a residence time ranging from about 5 minutes to about 20 minutes, with about 10 minutes to about 15 minutes as the optimal range and with a temperature ranging from about 132° C. to about 227° C. (about 270° F. to about 440° F.), with about 138° C. to about 210° C. (about 280° F. to about 410° F.) as the optimal temperature range. The high temperature water may help separate the components in the low-solids slurry stream. The pressure in the reactor 404 is the same as saturated steam pressure plus 25 psig, which is controlled by venting to flash tanks 406, 408, and 410. In an embodiment, some of the pretreatment 110 condensate from the flash tanks 406, 408, and 410 may be recycled to the starch ethanol plant 146.

The reactor 404 may include designing an agitator located near an edge of the reactor 404. The reactor 404 has upflow or downflow agitation, which agitates the low-solids slurry. The edge location of the agitator in the reactor 404 prevents fouling in the reactor 404. The material has been previously referred to as low-solids slurry or low-solids slurry stream, but will now be referred to as pretreated feedstock.

The pretreatment 110 sends the pretreated feedstock from the reactor 404 to one or more flash tank(s) 406, 408, 410. The reactor 404 releases the pretreated feedstock with an explosive decompression in one or more stages. The flash tank(s) 406, 408, 410 may each include an agitator with upflow or downflow, which agitates the pretreated feedstock. In an embodiment, there may be one or more flash tanks, such as a first flash tank 406, a second flash tank 408, and a third flash tank 410. The retention time of the pretreated feedstock in the flash tanks 406, 408, and 410 may range from greater than about 5 minutes to about 60 minutes. Each stage in a flash tank may be greater than about 5 minutes for each stage or the time may vary slightly from one flash tank to another flash tank. The flash pressure adjusts the temperature of the pretreated feedstock to about 40° C. to 104° C. (about 104° F. to about 220° F.) in the final flash tank 410 and the pressure ranges from about 1 psia to about 17 psia.

In an embodiment, pretreatment 110 further adjusts the pH of the pretreated feedstock by neutralizing it with a base 412 in the first flash tank 406 and/or the second flash tank 408 to about 3.5 to about 6. In other embodiments, pretreatment 110 adjusts the pH of the pretreated feedstock by neutralizing it with the base 412 in the first flash tank 406 or in the third flash tank 410. The pretreatment 110 adjusts the pH to greater than about 3 to less than 6. The base 412 helps with fermentation in the process 100 and in aerobic propagation. The base 412 that may be used include, but is not limited to, aqueous ammonia, anhydrous ammonia, sodium hydroxide, potassium hydroxide, calcium hydroxide, or any other bases. The calculations for the amount of ammonia are based on a mass balance and based on the amount needed in the aerobic propagation to convert carbon source to yeast.

Next, the pretreated feedstock undergoes hydrolysate conditioning. This occurs by adding more base to the pretreated feedstock. For example, the pretreatment 110 adjusts the pH to greater than about 4 with ammonia to provide the nitrogen source for yeast growth during aerobic propagation later in the process, that is for SCP production in aerobic propagation. The flash tanks 406, 408, 410 provides flash steam 414, 416, and 418 and the pretreated feedstock to be further processed in hydrolysis 111. In an embodiment, the evaporator condensate may come from the steam given off by the flash tank in the pretreatment 110. Having an efficient pretreatment may reduce the enzyme dosage in hydrolysis and enhance the yield of simple sugars. Examples of data are illustrated in tables towards the end of the description.

Hydrolysis of Pretreated Feedstock

FIG. 5 illustrates an example process of hydrolysis 111. Hydrolysis 111 converts a majority of the Pretreated Feedstock 502 from cellulose and hemicellulose to glucose and xylose with a cellulase enzyme. Hydrolysis 111 may use base and cellulase enzymes in combination with one or more viscosity break tank(s) and one or more hydrolysis tank(s) to maximize yield increase.

Hydrolysis 111 receives the Pretreated Feedstock 502 from the flash tank 410 of pretreatment 110 in one or more viscosity break tank(s) 112(A), 112(B). The pretreatment 110 opened the materials to increase enzyme accessibility while minimizing sugar loss. Next, hydrolysis 111 adds base to the Pretreated Feedstock 502 in the first viscosity break tank 112(A), second viscosity break tank 112(B) and hydrolysis tank(s) 114(A)-(D). Most of the base is added in the first viscosity break tank 112(A) for pH control. There may be one or more viscosity break tanks depending on variables such as capacity of the processes, the percent solids, the size of the tanks, and such. The viscosity break tanks may include an agitator with upflow or downflow, which agitates the Pretreated Feedstock 502. Hydrolysis 111 adds enzymes 506 to one or more viscosity break tank(s) 112 and/or to one or more hydrolysis tank(s) 114. Following enzyme addition to the viscosity break tanks, the material is referred to as Hydrolysate.

Converting cellobiose by β-glucosidases is a key factor for reducing cellobiose inhibition and enhancing the efficiency of cellulase enzymes for producing cellulosic biofuel. Cellobiose is a water-soluble disaccharide with two glucose molecules linked by β(1→4) bonds, which is obtained by breakdown of cellulose upon hydrolysis. β-glucosidase is a glucosidase enzyme which acts upon β(1→4) bonds linking two glucose or glucose-substituted molecules, such as cellobiose.

The five general classes of cellulase enzymes include endocellulase, exocellulase, cellobiase, oxidative cellulases, and cellulose phosphorylases. Beta-1,4-endoglucanase is a specific enzyme that catalyzes the hydrolysis of cellulose. β-glucosidase is an exocellulase with specificity for a variety of beta-D-glycoside substrates. It catalyzes the hydrolysis of terminal non-reducing residues in beta-D-glucosides with release of glucose. The cellulase enzyme may include, but is not limited to, commercial products such as Novozymes CTec2. Novozymes CTec3, and the like.

In an embodiment, hydrolysis 111 adds a cellulase and hemicellulase complex enzyme that degrades the cellulose and hemicellulose to fermentable sugars, to the first viscosity break tank 112(A) and adds greater than 90% of the cellulase and hemicellulase complex enzyme to the second viscosity break tank 112(B).

In yet another embodiment, hydrolysis 111 adds a cellulase and hemicellulase complex enzyme that degrades the cellulose and hemicellulose to fermentable sugars, to the first viscosity break tank 112(A), the second viscosity break tank 112(B), and to the hydrolysis tank(s) 114(A)-(D).

Hydrolysis of the Pretreated Feedstock 502 occurs in the temperature range of about 40° C. to about 60° C. and adjusts the pH of the Pretreated Feedstock 502 to about 4.2 to 6 in the first viscosity break tank 112(A).

After the viscosity break tanks 112, the process stream goes through the hydrolysis tanks 114. The number of hydrolysis tanks may range from one to six tanks. In an embodiment, there are four hydrolysis tanks 114(A)-(D). The temperature range of the hydrolysate may be about 50° C. to about 60° C. (120° F. to about 140° F.) and the pH is in the range of 4 to 5.5 in the hydrolysis tanks. The process 111 sends the stream from the hydrolysis tank(s) to the first solid/liquid separation 116.

Fermenting, Separating, and Distilling Materials

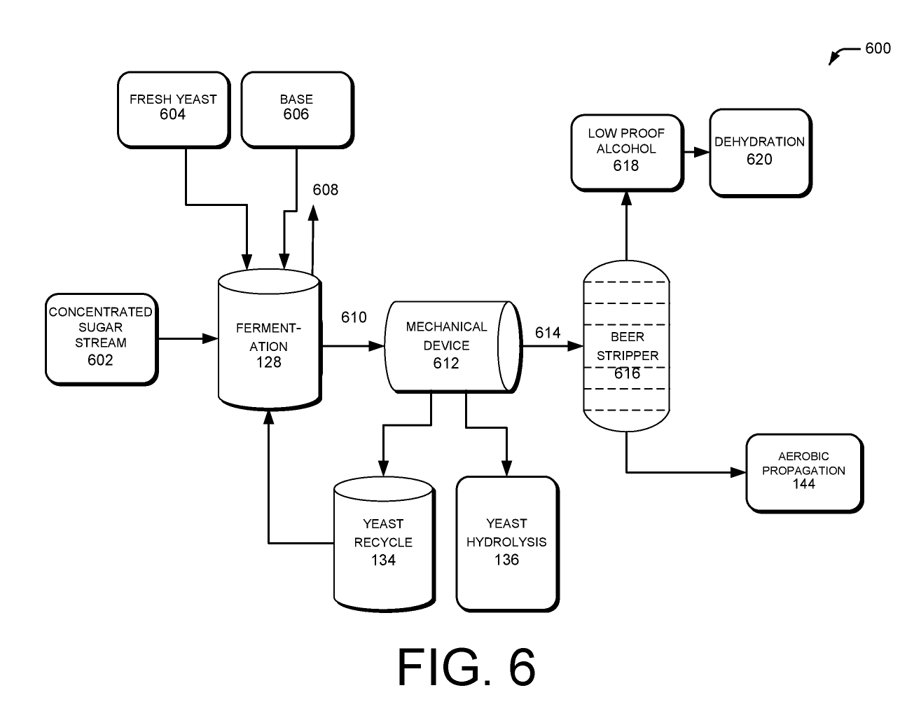

FIG. 6 illustrates an example process 600 to separate fermented materials after fermentation 128 for yeast recycle 134, yeast hydrolysis 136 and to distillation 142 beer to generate stillage for aerobic propagation 144. The process 600 sends the concentrated sugar stream 602 from evaporation 124 to fermentation 128, which becomes fermented to beer as described above. The process 600 adds fresh yeast 604 and base 604 to fermentation 128 while releasing carbon dioxide 608. The process 600 sends the beer 610 containing about 3% to about 5% yeast w/w and about 4% to about 8% alcohol w/w through a mechanical device 612, which may be used as the second solid/liquid separation 132. The mechanical device 612 creates yeast solids at about 12% to about 35% suspended solids (greater than 50% viability in yeast): a first yeast solids and a second yeast solids, and clarified beer at about 0.1% to about 4% suspended solids. The mechanical device 612 may be a disc stack centrifuge, a nozzle centrifuge, a sedi-canter, a membrane separation device, a washing stage included with the mechanical device, and the like. In another embodiment, the mechanical device may generate only two streams: a single yeast solids and clarified beer.

The process 600 sends the first yeast solids to yeast recycle 134 to condition yeast for reuse as catalyst for anaerobic fermentation. The mechanical device 612 may include a washing stage or the process 600 may include a washing mechanism that applies a chemical to remove contaminant organisms from the first yeast solids. The chemical may include, but is not limited to, a low pH solution of less than 3.5, chlorine dioxide, sulfite, sulfuric acid, used alone or in combination. Washing helps to decrease the amount of chemical needed in yeast recycle 134 and to maintain a more viable yeast. The washing would also retain more sulfur within the cellulosic biofuel plant when generating Co-Products B 141. In an embodiment, the process 600 adds sulfuric acid to the first yeast solids at about 8° C. to about 12° C. (about 46° F. to about 54° F.) for about 8 minutes to about 120 minutes of washing. The process 600 sends the recycled yeast stream from yeast recycle 134 to be reused in fermentation 128.

In another embodiment, the process 600 provides the first yeast solids with nutrient sources (i.e., fermentation feed, starch sugars, and the like), adjusts the pH to about 5 to about 6 by adding acid or a base, provides air, and adds sugar to improve viability prior to recycle 134. This embodiment may occur at about 28° C. to about 32° C. (about 82° F. to about 90° F.) for about 1 hour up to 24 hours.

The process 600 sends the second yeast solids to yeast hydrolysis 136. Details of yeast hydrolysis 136 are described with reference to FIGS. 8 and 9.

Next, the process 600 sends clarified beer 614 to a beer stripper 616 in which the product with the lowest boiling point, such as low proof alcohol 618 leaves the top of the beer stripper 616 in a vapor form, and the product with the highest boiling point, such as cellulose stillage exits from the bottom of the beer stripper 616. The process 600 sends the product with the highest boiling point to aerobic propagation 144, which is discussed with reference to FIGS. 10 and 11.

In an embodiment, the low proof alcohol 618 goes to a rectifier column, which creates 180 to 190 proof alcohol. The process 600 may send the 180 to 190 proof alcohol vapor through a condenser for cooling and to convert to a liquid form. The process 600 may send the bottom liquid from the rectifier column into a side stripper column, which strips the alcohol from the water and adds it back into the rectifier column. This stream may be used as water in pretreatment 110 or as cook water in the starch ethanol plant 146. Then the 180-190 proof alcohol 618 goes through dehydration 620.

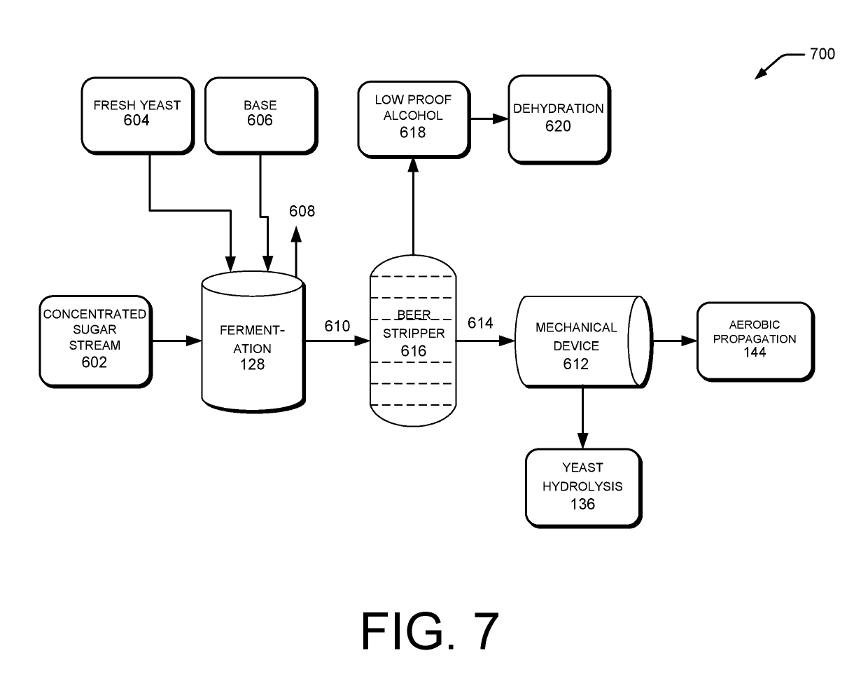

FIG. 7 illustrates an example process to separate fermented materials for yeast hydrolysis, and aerobic propagation. The processes in FIG. 7 that are similar to the processes described with reference to FIG. 6, will not be described again. The process 700 shows no yeast recycle and a different order of equipment than what was shown in FIG. 6.

Yeast Hydrolysis

FIGS. 8 and 9 illustrate example processes of yeast hydrolysis 136 to produce cellulosic biofuel 1143 and Co-Products B 141, such as single-cell protein (SCP). The purpose of yeast hydrolysis 136 is to hydrolyse the yeast by enzymes and/or heat to produce SCP. The SCP produced may be used in animal feed product, has an amino acid profile that is comparable to animal feed products currently sold in the market.

FIG. 8 illustrates an example process 800 of yeast hydrolysis 136 that receives the yeast solids 802 after fermentation 128 in which a mixture of enzymes 804 were supplied to the yeast solids 802 and after separation by a mechanical device 612. The mixture 804 may include, but is not limited to, a mixture of enzymes such as proteases, amylases, cellulases, and the like. The mixture 804 helps to break viscosity and increases protein digestibility in animal feed rations. The process 800 adjusts the pH to below about 6 in the tank 806, keeps the temperature in a range of about 43° C. to about 54° C. (about 110° F. to about 130° F.), and has a retention time of about 1 to about 24 hours in the tank 806. Variables that affect pH, temperature, and time include the types of enzymes in the mixture chosen as well as the types of biomass feedstock.

Returning to tank 806, the process 800 sends the hydrolyzed yeast 808 to recovery evaporators 138 to minimize drying costs and to recover alcohol. The recovery evaporators 138 concentrate the hydrolyzed yeast 808 to generate a concentrated yeast 810 at about 30% to about 80% solids and a low proof alcohol 812. One skilled in the art would expect to add water and/or steam to the recovery evaporators 138 and to release condensate from the recovery evaporators 138. The process 800 further takes the low proof alcohol 812 through distillation 142 and dehydration to produce cellulosic biofuel 143.

In an embodiment, the process 800 may include the concentrated yeast 810 as part of animal feed to be blended into high protein Dried Distillers Grain with Solubles (DDGS). Furthermore, the concentrated yeast 810 may be used internally in the process as yeast extract or sold to third parties as yeast extract.

In another embodiment, the process 800 may send the concentrated yeast 810 to be dried in yeast dryer 140 to become even more concentrated, at greater than about 85% solids. The yeast dryer 140 may include, but is not limited to, a spray dryer, a fluid bed, a ring dryer, a yeast dryer, and the like. This produces Co-Products B, 141, such as single cell protein 814, which may be sold as animal feed. SCP 814 may have protein levels over 35% by weight, an amino acid profile that is similar to products produced from brewer's yeast, and a total digestible nutrient greater than 80%. Lab data of SCP 814 are shown in the Examples.

FIG. 9 illustrates another example of the yeast hydrolysis 136 to produce cellulosic biofuel 143 and single cell protein 814. The processes in FIG. 9 that are similar to the processes in FIG. 8 will not be described again. FIG. 9 illustrates another embodiment of yeast hydrolysis 136. The process 900 shows that the process stream from the recovery evaporators 138 may be sent to evaporator condensate 123 and/or cook water 158.

Aerobic Propagation

FIGS. 10 and 11 illustrate example processes of aerobic propagation 144 to produce ethanol producing catalyst and co-products in the cellulosic biofuel plant. A cellulosic biofuel plant may receive yeast from a supplier or may choose to propagate yeast, which is growing the yeast needed for fermentation 128. FIG. 10 illustrates the process 100 of aerobically propagating a fermenting yeast on a culture medium to maximize production of ethanol producing catalyst and co-products. Aerobic propagation 144 reproduces the fermenting yeast by using its own natural capabilities as living organisms. However, aerobic propagation 144 needs a carbon source, aeration, and nutrients for the fermenting yeast.

In an embodiment of a continuous mode, the process 1000 receives a first amount of process stream of cellulosic stillage 1002 from distillation 142 of the cellulosic biofuel plant and a second amount of process stream of stillage 1004 from the starch ethanol plant 146 into a tank 1006. The amounts of stillages from each of the plants may vary from about 1% to about 99% depending on a ratio of the starch and cellulosic process rates. The cellulosic stillage 1002 may be concentrated or non-concentrated stillage. In embodiments, the process stream of stillage 1004 from the starch ethanol plant 146 may be a defatted concentrated starch stillage stream, which may be optimally clarified and concentrated, with majority of oil removed and solids, or non-clarified, with oil and solids. In another embodiment, the process stream of stillage may be sugar cane stillage (e.g., vinasse) from a sugar cane plant. The process stream may be from different sources based on its source plant being located adjacent to the cellulosic biofuel plant.

The culture medium that the fermenting yeast can grow on may provide the carbon source. The culture medium may include soluble proteins, carbohydrates, organic acids, fats, inorganic micronutrients and macronutrients, and the like. Propagation may be continuous, batch, or semi-continuous.

Next, the process 1000 adds a base 1007 such as a waste clean in place to tank 1006. Microbial contamination may be a problem in aerobic propagation 144. Thus, the process 1000 may send the combined two process streams 1002, 1004 with the base 1007 through a continuous sterilization process, prior to a propagation tank. The continuous sterilization process may include indirect heat exchange or direct steam injections. In another embodiment, the process 1000 sends each of the process streams 1002, 1004 individually through a sterilization process prior to the propagation tank.

Next, the process 1000 adds a yeast 1008 to the cellulosic stillage 1002 combined with the stillage 1004 and base 1007 as a culture medium, into propagation tank 1010. In another embodiment, the process 1000 starts with the starch stillage 1004, adds yeast 1008 to the starch stillage 1004, and then adds the cellulosic stillage 1002. The process should not be construed as necessarily order dependent. Any number of the described processes may be combined in any order to implement the method, or an alternate method.

Yeast 1008 is a fermenting yeast, which may include, but is not limited to, a GMO yeast, a C5/C6 GMO yeast, a GMO Saccharomyces cerevisiae yeast, a Saccharomyces cerevisiae yeast, an ethanol fermenting, and aerobic glycerol consuming yeast, and the like. The process 1000 may include one to ten propagation tanks, 1010, 1012, 1014, 1016, which may be an airlift tank or an agitated tank about 2% to about 15% of the size of an ethanol fermentor in fermentation 128.

The GMO yeast anaerobically converts the C5/C6 sugars to ethanol while also being capable of aerobically converting stillage components (primarily glycerol) to yeast mass efficiently. Genetic modifications may be made to a naturally occurring host yeast that efficiently converts stillage components (primarily glycerol) to yeast cell mass. The genetic modifications allow for both aerobic conversion of glycerol to yeast mass and genetic modifications that allow for efficient anaerobic fermentation of C5/C6 sugars to ethanol.

The process 1000 inoculates yeast 1008 at time 0 or start of fill to be calculated as part of the working volume of the propagation tank 1010, to about 1 to 3E10⁷ colony-forming unit/milliliter (cfu/ml). The culture medium may exhaust all of the carbon sources as the culture medium leaves the last propagation tank, causing the process 1000 to add mixture of starch stillage 1004 and cellulosic stillage 1002 to one of the propagation tanks. The process 1000 transfers the culture medium actively from one propagation tank to another or by overflowing from one propagation tank to another propagation tank. The process 1000 may add air to the propagation tanks.

The cellulosic stillage 1002 contains high concentration of organic components, such as glycerol, acetate, lactate, and residual sugars. The cellulosic stillage 1002 also contains high concentration of inorganic components such as nitrogen obtained from ammonia used in neutralization or hydrolysis processes. The nitrogen would serve as additional nutrients for the yeasts to optimize growth. The amount of ammonia is determined by the requirements for aerobic propagation. The ammonia used in neutralization is ultimately converted to yeast cell mass in aerobic propagation of mixed stillage. The yeast converts ammonia to protein, which yeast cells are made of 50% protein.

Adding low cost carbon sources such as glycerine water from biodiesel production into the mixed stillage stream to increase the concentration of aerobically convertible carbohydrates will increase the amount of yeast produced in the process 1000.

The requirements of aerobic propagation are driven by the amount of convertible carbohydrate in the combined stillage stream. The concentration of convertible carbohydrate in the combined stillage stream is a function of the size of the starch ethanol plant to cellulosic ethanol plant based on stillage blend rates.

The operating conditions for optimal aerobic propagation 144 in the propagation tanks 1010, 1012, 1014, and 1016 include a comfortable temperature for growing and metabolism of yeast ranging from about 25° C. to about 40° C. (about 77° F. to about 104° F.). Higher temperatures create stress compounds and reduces reproduction while lower temperatures result in slow metabolism and reproduction. Other optimal conditions include: a pH ranging from about 3 to about 8, a pressure at about 1 to about 30 psig, aeration provided from atmospheric concentration air to oxygen enriched air (about 20 to about 100% w/w), dissolved oxygen controlled from 1-10 ppm in propagation tank 1010 by controlling agitation and aeration rates, and adequate time for reproduction ranging from about 10 hours to about 70 hours, depending on the types of yeasts, culture media, and media composition. The process 1000 may need to add a known feed-grade antifoam into the tank 1006 or any of the propagation tanks to control foaming due to the added air and media composition. The pH may be controlled by acid and/or base, such as sulfuric acid, phosphoric acid, hydrochloric acid, waste CIP, and the like into mixed stillage tank 1006 or propagation tanks 1010, 1012, 1014, and 1016. The operating conditions may vary depending on the species of the yeasts and the culture medium.

The aerobic propagation 144 continues until the desired yeast population is reached or until almost most of the carbohydrate is converted to yeast cell mass.

After aerobic propagation 1016, the process 1000 sends the culture medium with yeast 1008 through a mechanical separation device 1018 to separate the solids from the liquids. The process stream containing solids becomes concentrated into yeast paste 1020, a cream-like substance with about 12% to about 33% dry solids. The process 1000 may send the yeast paste 1020 from the mechanical separation device 1018 directly to be used in ethanol fermentation 128. In another embodiment, the yeast paste 1020 may be cooled and stored in separate, refrigerated cream tank prior to use in ethanol fermentation 128. In another embodiment, a fraction of the yeast may be sent to ethanol fermentation 128, while another fraction will be sent as single cell protein (SCP) in the system (yeast hydrolysis tank 806). The mechanical separation device includes, but is not limited to, a decanter, a disk stack centrifuge, a membrane filtration system, a dynamic cross flow filtration, a dual-stage centrifugation, a combination of a centrifuge and a polishing device, and the like.

The liquids include yeast centrate 1022, which contains majority remaining biochemical oxygen demand (BOD), sulfate, and other soluble components. The process 1000 sends the yeast centrate 1022 through methanation 152 in which a methanator converts the BOD and sulfate, to methane and hydrogen sulfide, respectively. The methanator also produces additional energy and removes sulfur (SOx reducation) from the yeast centrate stream. In a two phase methanation system, the process 1000 uses a two phase acidogenic/methanogenic technology to treat the yeast centrate 1022 from a cellulosic biofuel plant. The process would be acidogenic followed with methanation.

FIG. 11 illustrates another example of the aerobic propagation 144 to produce co-products. The processes in FIG. 11 that are similar to the processes in FIG. 10 will not be described again. FIG. 11 illustrates another embodiment.

FIG. 11 illustrates the process 1100 shown with a mechanical device 1102. In an embodiment, the mechanical device 1102 clarifies a defatted concentrated stillage stream 1103 from the starch ethanol plant 146 by removing almost most of the suspended solids from the stillage stream. The process 1100 combines the clarified stillage stream 1108 and the cellulosic stillage stream 1004 into the tank 1006, and adds a base to create a mixture in the tank 1006. Next, the process 1100 sends the mixture to a propagation tank 1010, where a yeast 1008 is added to process. There may be one or more propagation tanks. This creates co-products as shown.

The mechanical device 1102 may include, but is not limited to a paddle screen, a centrifuge, a decanter, a disk stack centrifuge, a membrane filtration system, a dynamic cross flow filtration, a dual-stage centrifugation, a combination of a centrifuge and a polishing device, any type of device capable of separating suspended solids from liquids. In another embodiment, the process 1100 receives a stillage stream 1004 from the starch ethanol plant 146, sends the stillage stream 1004 through a mechanical device 1102 to remove suspended solids 1106 to become clarified stillage 1108. Hereinafter, the process 1100 performs similar actions in FIG. 11 that are similar to the processes described with reference in FIG. 10. As mentioned, the streams may be stillage 1004 from the starch ethanol plant or a defatted concentrated stillage stream 1103.

Examples with Results

The examples below are only representative of some aspects of this disclosure. It will be understood by those skilled in the art that processes as set forth in the specification can be practiced with a variety of alterations with the benefit of the disclosure. These are examples and the procedures used therein should not be interpreted as limiting the invention in any way not explicitly stated in the claims.

Wash Data

An experiment was performed for washing feedstock. Switchgrass (SG) as the feedstock was ground to 4 mm with a Retch mill prior to processing. Approximately 500 g of ground SG was washed with 6 L of hot tap water. Washed and unwashed feedstocks of SG were then evaluated via NREL-LAP for compositional analysis.

TABLE
Washed Feedstock

| | | |
|---|---|---|
| 93% | 88% | W H_M C |
| 92% | 92% | W M TS as received |
| NA | NA | W M Protein |
| 11% | 8% | W M Total Ext |
| 9% | 6% | W M H20 Ext |
| 2% | 2% | W M EtOH Ext |
| 2% | 2% | W M ASL |
| 14% | 13% | W M AIL |
| 5% | 3% | W M Ash |
| NA | NA | W M Sucrose |
| NA | NA | W M Acetyl |
| 2% | 2% | W M Mannan |
| 2% | 2% | W M Arabinan |
| 2% | 2% | W M Galactan |
| 20% | 21% | W M Xylan |
| 33.4% | 35.0% | W M Glucan |
| unwashed | washed | description |
| B0078-02-001 | B007-02-000 | Experment ID |

This data shows an increase in carbohydrates of 59.4 to 62% (4.4% increase) due to removal of 2% ash and 3% water extractives. Following washing the feedstock was pretreated in a lab scale reactor generating between 80-100 g of pretreated material. The total solids in the test was 13%, temperature 160-190° C., sulfuric acid dosed at 4-5% of the feedstock dry matter, retention time 4-16 minutes, flash cooling to stop reaction.

Following pretreatment, slurries were pH adjusted to 5.2 with 1:1 w/w NaOH:KOH mixture. In the Hydrolysis vessels, tetracycline (8 ppm) was added to control contamination along with equivalent (5 mg enzyme protein/g cellulose) cellulase (CTEC2) dosing. Every 24 hours, a sample was pulled for HPLC analysis to track sugar hydrolysis. At 120 hours, samples were tested for solids profiling and percentage sugar conversions calculated. A significant increase in sugar hydrolysis was noted for the washed vs. the unwashed substrates.

Use of Cellulose Wash Water in Starch Ethanol Plant

A 34% as-is slurry of Lifeline Food endosperm was prepared by using 40% of the makeup water being thin stillage and varying amount of tap water and Energy Sorghum Wash Water (ESW) such that the amount of ESW varied from 0 to 60% of the makeup water. The 60% ESW level was essentially the highest level of ESW that could be use, which meant no tap water was used only ESW and thin stillage. The slurry was adjusted to pH 5.6-5.8 using 10% sodium hydroxide. Alpha-amylase (Liquozyme SC DS from Novozymes) was added at 0.02% of the slurry solids and then liquefied at 85° C. for two hours. The mash was then cooled to 32° C. and the pH adjusted to 4.8 with sulfuric acid. A sample of the mash was then taken for solids determination, brix, DE and HPLC analysis.

The mash was then prepared for fermentation by adjusting the pH to 4.8, adding 0.7 kg/MT of mash solids gluco-amylase, 0.3 kg/MT of protease (Fermgen from Genencor), 900 ppm urea, and 1 ppm antibiotic (Bactinex V60 from NABC). The mash was then dry pitched at 0.1% with active dry yeast (Bio-Ferm XR from NABC). The mash was stirred for about 10 minutes, and then triplicate flasks were prepared by adding 300 gm of mash to 500 ml Erlenmeyer flasks. The flasks were sealed with a rubber stopper containing an 18 gauge needle to vent the flask and then placed in temperature control rotary shaker set at 150 rpm and 32° C. At 6, 24, 48 and 70 hours samples were removed from the flasks for HPLC analysis. Another set of fermentors were prepared in triplicate by adding 150 gm of mash to tarred 250 ml Erlenmeyer flask. The flasks were sealed with a rubber stopper containing an 18 gauge needle and placed in the temperature controlled shaker at the conditions described above. When the 500 ml flasks were sampled, the 250 ml flasks were weight. In this manner, the fermentation in 250 ml flasks was monitored by weight loss. The weight loss was then used to calculate the amount of ethanol produced.

After 70 hours of fermentation the beer in the 250 ml flasks was transferred to 250 ml centrifuge bottle and centrifuged at 5000 rpm for 5 minutes. A sample of the supernatant was taken for HPLC analysis and the remainder of the supernatant discarded. The pellet was quantitatively as possible transferred to a weight boat and dried at 65° C. to obtain the DDG. The DDG samples were assayed for moisture, starch and protein.

Table 1 gives the HPLC carbohydrate profile of the ESW and thin stillage used in the mash make-up. Water resulting from washing of the cellulosic feedstock can be utilized effectively as cook water in the co-located starch ethanol plant.

TABLE 1
HPLC Profiles of ESW and Thin Stillage

| | | HPLC Profile (% W/V) | | | | | | | |
|---|---|---|---|---|---|---|---|---|---|
| Sample | % DS | DP4₊ | DP3 | Maltose | Glucose | Lactic | Glycerol | Acetic | Ethanol |
| ESW | 0.99 | 0.13 | BDL^{a} | BDL | BDL | 0.32 | 0.01 | 0.13 | 0.10 |
| Thin Stillage | 5.15 | 0.82 | 0.06 | 0.54 | 0.16 | 0.11 | 1.59 | 0.05 | BDL |

^{a}Below detection Limit

Table 2 summarizes some of the mash properties, and Table 3 shows the HPLC profiles of the mashes. The mash DE values are higher than what is required, and Mash B for some reason is unusually high. The HPLC profiles in Table 3 are quite similar with a little more lactic acid as the amount of ESW increased in the mash.

TABLE 2
Mash Properties

| Trial | % ESW | % DS | Brix | DE | Viscosity^{a} |
|---|---|---|---|---|---|
| A | 0 | 29.85 | 26.0 | 20.0 | 560 |
| B | 10 | 30.36 | 26.4 | 25.0 | 590 |
| C | 20 | 30.26 | 26.1 | 19.8 | 520 |
| D | 40 | 30.39 | 26.4 | 20.6 | 490 |
| E | 60 | 30.65 | 26.3 | 19.5 | 470 |

^{a}Viscosity measured with Brookfield viscometer at 32° as cp

TABLE 3
Mash HPLC Profile

| | | HPLC Profile (% W/V) | | | | | | | |
|---|---|---|---|---|---|---|---|---|---|
| Trial | % ESW | DP4₊ | DP3 | Maltose | Glucose | Lactic | Glycerol | Acetic | Ethanol |
| A | 0 | 27.84 | 2.82 | 1.56 | 1.07 | 0.09 | 0.50 | 0.00 | 0.00 |
| B | 10 | 26.88 | 3.05 | 1.84 | 1.24 | 0.16 | 0.56 | 0.10 | 0.00 |
| C | 20 | 26.85 | 3.17 | 1.99 | 1.26 | 0.18 | 0.56 | 0.11 | 0.03 |
| D | 40 | 26.63 | 3.18 | 2.02 | 1.29 | 0.22 | 0.55 | 0.09 | 0.03 |
| E | 60 | 26.74 | 3.22 | 2.07 | 1.32 | 0.29 | 0.56 | 0.15 | 0.04 |

Table 4 below summarizes the average fermentor HPLC profiles of the mashes. The results indicate that adding the ESW does not seem to negatively influence the fermentations. Actually the results show a slight increase in ethanol by the addition of ESW.

TABLE 4
Average Fermenter HPLC Profiles

| Trial | % ESW | Hour | DP4₊ | DP3 | Maltose | Glucose | Lactic | Glycerol | Acetic | Ethanol |
|---|---|---|---|---|---|---|---|---|---|---|
| A | 0 | 0 | 27.84 | 2.82 | 1.56 | 1.07 | 0.09 | 0.50 | 0.00 | 0.00 |
| A | 0 | 4 | 15.57 | 2.86 | 3.61 | 6.81 | 0.11 | 0.63 | 0.13 | 0.55 |
| A | 0 | 24 | 2.68 | 0.14 | 0.33 | 0.22 | 0.08 | 1.44 | 0.02 | 11.61 |
| A | 0 | 48 | 0.81 | 0.06 | 0.37 | 0.11 | 0.07 | 1.46 | 0.03 | 12.93 |
| A | 0 | 70 | 0.73 | 0.06 | 0.37 | 0.09 | 0.07 | 1.46 | 0.03 | 13.24 |
| B | 10 | 0 | 26.88 | 3.05 | 1.84 | 1.24 | 0.16 | 0.56 | 0.10 | 0.00 |
| B | 10 | 4 | 14.40 | 3.14 | 3.98 | 7.09 | 0.11 | 0.59 | 0.10 | 0.57 |
| B | 10 | 24 | 2.70 | 0.19 | 0.33 | 0.34 | 0.10 | 1.45 | 0.02 | 11.60 |
| B | 10 | 48 | 0.78 | 0.07 | 0.37 | 0.12 | 0.10 | 1.46 | 0.03 | 12.94 |
| B | 10 | 70 | 0.70 | 0.06 | 0.37 | 0.10 | 0.08 | 1.46 | 0.03 | 13.23 |
| C | 20 | 0 | 26.85 | 3.17 | 1.99 | 1.26 | 0.18 | 0.56 | 0.11 | 0.03 |
| C | 20 | 4 | 13.70 | 3.14 | 4.41 | 7.71 | 0.15 | 0.62 | 0.14 | 0.60 |
| C | 20 | 24 | 2.58 | 0.20 | 0.35 | 0.42 | 0.13 | 1.44 | 0.03 | 11.77 |
| C | 20 | 48 | 0.82 | 0.07 | 0.39 | 0.12 | 0.12 | 1.46 | 0.03 | 13.05 |
| C | 20 | 70 | 0.75 | 0.06 | 0.39 | 0.10 | 0.11 | 1.46 | 0.04 | 13.31 |
| D | 40 | 0 | 26.63 | 3.18 | 2.02 | 1.29 | 0.22 | 0.55 | 0.09 | 0.03 |
| D | 40 | 4 | 13.52 | 3.16 | 4.46 | 7.85 | 0.20 | 0.60 | 0.16 | 0.55 |
| D | 40 | 24 | 2.67 | 0.22 | 0.35 | 0.53 | 0.17 | 1.40 | 0.03 | 11.70 |
| D | 40 | 48 | 0.85 | 0.07 | 0.38 | 0.13 | 0.16 | 1.41 | 0.03 | 13.08 |
| D | 40 | 70 | 0.77 | 0.06 | 0.37 | 0.11 | 0.15 | 1.41 | 0.03 | 13.36 |
| E | 60 | 0 | 26.74 | 3.22 | 2.07 | 1.32 | 0.29 | 0.56 | 0.15 | 0.04 |
| E | 60 | 4 | 12.84 | 2.95 | 4.76 | 8.37 | 0.26 | 0.61 | 0.19 | 0.60 |
| E | 60 | 24 | 2.59 | 0.21 | 0.35 | 0.58 | 0.22 | 1.36 | 0.04 | 11.86 |
| E | 60 | 48 | 0.89 | 0.07 | 0.38 | 0.14 | 0.21 | 1.37 | 0.04 | 13.21 |
| E | 60 | 70 | 0.80 | 0.06 | 0.36 | 0.12 | 0.20 | 1.37 | 0.05 | 13.49 |

Table 5 summarizes the average amount ethanol in the fermentors calculated from fermentor weight loss. FIG. 1 summarizes the ethanol yield, and shows an increase in ethanol as more ESW was added to the mash. The ethanol yield from the fermentor weight loss results were normalized to the amount of endosperm obtaining a yield of ml of ethanol per kg of endosperm solids, which is given in Table 6. The results are interesting in that adding ESW does not seem to inhibit the fermentation rather there appears to be a slight increase in ethanol from the starch and or fermentable sugars in the ESW.

TABLE 5
Average Final Ethanol From Fermenter Weight Loss

| Trial | % ESW | Ethanol (% W/W) | Stdev |
|---|---|---|---|
| A | 0 | 11.84 | 0.00 |
| B | 10 | 11.82 | 0.01 |
| C | 20 | 11.92 | 0.01 |
| D | 40 | 11.94 | 0.03 |
| E | 60 | 12.04 | 0.01 |

TABLE 6
Average Ethanol Yield from Fermenter Weight Loss

| Trial | % ESW | Yield^{a} | Stdev | % Incre. |
|---|---|---|---|---|
| A | 0 | 479.4 | 0.1 | 0.0 |
| B | 10 | 478.6 | 0.3 | −0.2 |
| C | 20 | 482.0 | 0.4 | 0.6 |
| D | 40 | 482.9 | 0.8 | 0.7 |
| E | 60 | 486.6 | 0.2 | 1.5 |

^{a}Yield as ml of ethanol per kg of endosperm DS.

The amount of DDG from each mash was calculated as gm of DDG solids per kg of endosperm solids, and is summarized in Table 7. The solids in ESW was low (0.99%) and did not seem to contribute to the amount of DDG recovered.

TABLE 7
DDG Yield (gm DDG/kg Endosperm)^{a}

| Trial | % ESW | Average | Stdev |
|---|---|---|---|
| A | 0 | 264.7 | 0.7 |
| B | 10 | 266.7 | 1.9 |
| C | 20 | 257.5 | 5.7 |
| D | 40 | 262.8 | 3.8 |
| E | 60 | 264.9 | 4.9 |

^{a}DDG and Endosperm as DS

Table 8 summarizes the starch and protein composition of the DDG from each of the mashes after fermentations. The starch content seems to decrease a little by the addition of ESW, and the protein content seemed to decrease slightly with the addition of ESW, which probably is insignificant.

TABLE 8
DDG Starch and Protein Composition

| | |
|---|---|
| % Starch (dsb) | % Protein (dsb) |

| Trial | % ESW | Average | Stdev | Average | Stdev |
|---|---|---|---|---|---|
| A | 0 | 10.04 | 0.12 | 33.11 | 0.06 |
| B | 10 | 10.35 | 0.07 | 32.61 | 0.24 |
| C | 20 | 8.93 | 0.21 | 33.36 | 0.43 |
| D | 40 | 9.32 | 0.19 | 32.87 | 0.20 |
| E | 60 | 9.13 | 0.06 | 32.57 | 0.30 |

### Pretreatment Condensate on Corn Mash Fermentation Example

The main objective was to determine to what amount of bran pretreatment condensate (PC) can be added as make-up water in corn mash that would not be detrimental to ethanol yield. Corn bran PC was obtained from ICM's pilot plant. The experiment used a 2 L glass reactor, added 720 g of corn flour, 704 g cook water and 576 g of backset all from Lifeline Foods. The pH of the slurry was adjusted to 5.5, and alpha-amylase was added to the slurry at 0.02% of corn solids. The slurry was heated to 85° C. and held at 85° C. for one hour, and milled on high setting for one minute in a 4 L Waring blender. The mash was then held at 85° C. for another hour and then adjusted to pH 4.8 and cooled, and stored in the cooler until used for fermentation. A series of liquefaction were also conducted in a similar manner except the cook water was replaced at various percentages of 10%, 25%, 40%, 70% and 100% with PC.

The mashes were prepared for fermentation by warming to room temperature and then adding gluco-amylase at 0.06% of corn solids, protease at 0.03% of corn solids, 600 ppm urea (based on mash weight), and 1 ppm antibiotic. The mash was then inoculated at 0.1% (w/w) with active dry yeast. The mash was stirred for about 10 minutes, and triplicate flasks were prepared by adding 150 g of mash to 250 ml Erlenmeyer flasks. The flasks were sealed with a rubber stopper containing an 18 gauge needle, and placed in a temperature controlled shaker/incubator set at 32° C. and 150 rpm. At 6, 16, 25, 48 and 70 hours, samples were removed from the flasks for HPLC analysis. After sampling, the samples were immediately incubated at in 75° C. water bath to inactivate the enzymes prior to preparing the samples for HPLC analysis. Another set of fermentors were prepared in triplicate by adding 150 gm of mash to tarred 250 ml Erlenmeyer flask. The flasks were sealed with a rubber stopper containing an 18 gauge needle and placed in the temperature controlled shaker at the conditions described above. When the first set of fermentors were sampled for HPLC the second set of fermentors were weight. The weight loss results from the second set of fermentations was used to calculate ethanol level as % w/w. After 70 hours of fermentation, beer from the HPLC flasks was discarded. After 70 hours, a sample of the weight loss fermentors was removed for HPLC analysis.

TABLE 9
Average Final Ethanol (% w/v) by HPLC

| Trial | % PC | Hours | Ave | Stdev | Rel |
|---|---|---|---|---|---|
| A | 0 | 70 | 12.27 | 0.08 | 100.0 |
| B | 10 | 70 | 12.42 | 0.01 | 101.2 |
| C | 25 | 70 | 12.54 | 0.01 | 102.2 |
| D | 40 | 70 | 12.65 | 0.02 | 103.1 |
| E | 70 | 70 | 12.53 | 0.01 | 102.1 |
| F | 100 | 70 | 12.75 | 0.02 | 103.9 |

Trials A-F show ranges of % PC at 0, 10, 25, 40, 70, and 100% and ethanol about 100% and 103.9% weight per volume (w/v). The last column for ethanol yield data (relative values to Trial A) shows an increase based on increased percentages of PC. For instance, ethanol yield increased ranging from 1% to almost 4%. High-performance liquid chromatography (HPLC) results showed that as the amount of PC is at the 100% level, a gradual increase in ethanol yield occurred to about a 4% increase.

TABLE 10
Average Final Ethanol Yield Calculated From Fermentor Weight Loss.

| Trial | % PC | % DS | % W/W | Stdev | Rel | g/kg^{a} | Stdev | Rel |
|---|---|---|---|---|---|---|---|---|
| A | 0 | 31.55 | 11.58 | 0.02 | 100.0 | 328.5 | 0.5 | 100.0 |
| B | 10 | 31.67 | 11.60 | 0.03 | 100.1 | 327.6 | 0.6 | 99.7 |
| C | 25 | 31.78 | 11.66 | 0.01 | 100.6 | 328.0 | 0.3 | 99.9 |
| D | 40 | 31.99 | 11.79 | 0.07 | 101.8 | 329.1 | 1.7 | 100.2 |
| E | 70 | 31.50 | 11.61 | 0.01 | 100.3 | 329.7 | 0.2 | 100.4 |
| F | 100 | 31.64 | 11.80 | 0.00 | 101.8 | 332.9 | 0.1 | 101.3 |

^{a}Ethanol yield calculated as g of ethanol per kg of mash dry solids

TABLE 11
Average DDGS Composition

| | | % Starch (dsb) | | % Protein (dsb) | | % Oil (dsb) | |
|---|---|---|---|---|---|---|---|
| Trial | % PC | Ave | Std | Ave | Std | Ave | Std |
| A | 0 | 3.47 | 0.01 | 29.36 | 0.09 | 18.86 | 0.44 |
| B | 10 | 3.36 | 0.02 | 30.14 | 0.33 | 19.41 | 0.45 |
| C | 25 | 3.15 | 0.02 | 30.67 | 0.10 | 19.60 | 0.89 |
| D | 40 | 3.14 | 0.04 | 30.13 | 0.20 | 18.68 | 0.09 |
| E | 70 | 3.04 | 0.02 | 30.52 | 0.22 | 18.70 | 0.16 |
| F | 100 | 3.05 | 0.01 | 30.13 | 0.19 | 19.13 | 0.49 |

Use of condensate resulting from the flashing of the pretreated cellulosic feedstock as cook water in the co-located starch ethanol plant.

Pretreatment was operated at demonstration scale (7-8 tons/day) utilizing switchgrass as the feedstock in a continuous pretreatment system. Water sources utilized were stillage evaporator condensate from a co-located 50 million gallon per year starch to ethanol plant and evaporator condensate from the concentration of switchgrass sugars prior to fermentation. Switchgrass was washed with hot water prior to being slurried. The switchgrass slurry was then sent through a pump to bring the slurry to pretreatment pressure. Following the pressurization of the switchgrass slurry, sulfuric acid was injected into the system. The slurry containing the sulfuric acid catalyst was then passed through the pretreatment reactor where temperature was controlled by live steam injection. Following the pretreatment residence time the slurry was flashed and pH adjusted in the flash tank with ammonium hydroxide (see post flash slurry chart). The ammonium hydroxide utilized in pH adjustment of the post flash slurry is ultimately utilized for yeast growth in the aerobic propagation (reference Aerobic propagation table showing ammonia consumption). To show pretreatment efficacy the change in composition of structural sugars is presented along with monomeric sugar composition in post flash slurry. This data shows that the xylan portion of the feedstock was dissolved into the soluble monomeric phase (see decrease in xylan in suspended solids and xylose concentration on graph) and the glucan concentration was increased in the suspended solids (table) with a very small increase in monomeric glucose concentration in the post flash slurry. This slurry was passed forward to hydrolysis for further enzymatic hydrolysis to monomeric sugars for fermentation.

Table of Changes in Concentration of Sugars in Suspended Solids
Concentration of Structural Sugars in Suspended Solids

| Description | Xylan % w/w | Glucan % w/w |
|---|---|---|
| Washed Feedstock | 17.8% | 31.6% |
| Post Flash Pretreated Slurry | 3.4% | 48.6% |

Water stream supply—Use of condensate water from fermentation feed evap to pretreatment condensate and use of condensate water from starch co-located water.

Injection of sulfuric acid after cellulosic slurry has reached pretreatment pressure, (this is for low solids PT, no soaking of biomass b/c not effectively mix). Look at drawing of pretreatment. Adding pH adjustments into mechanical agitated tank for low solids pretreatment, show increase in ammonia concentration with data.

Aerobic Propagation

De-oiled concentrated starch stillage and cellulosic stillage from switchgrass were aerobically propagated with a GMO yeast capable of aerobic propagation on stillage based components (glycerol primarily) and the stillage propagated yeast is capable of anaerobically producing ethanol from both 5 and 6 carbon sugars. Initially, the yeast was grown on a starch stillage only in batch phase and then mixed stillage was continuously fed into the fermentor in continuous mode. Two aerobic fermentors were run in series in continuous mode being fed with mixed stillage sterilized continuously. As shown in the figures below, the feed to the aerobic fermentors contained ˜2000 ppm ammonia, originating from flask tank pH adjustment in pretreatment, and on average the concentration in the continuous fermentors was maintained below 1000 ppm, which shows the culture converting ammonia to protein (cell mass). Similarly, the primary carbon source, glycerol, was present in the fermentor feed at 14-22 g/L and in the active aerobic fermentor the concentration was near zero for the majority of the run with a single upset around the 130 hour mark.

Data Charts of Consumption of Primary Components in Mixed Stillage and Yeast Concentration from Aerobic Propagation

| Cell Concentration | | | |
|---|---|---|---|
| cfu/ml | | 2.43E+088 | 28.2E+08 |
| Acetic Acid g/L | 4.94 | 0.38 | 0.05 |
| Lactic Acid g/L | 3.54 | 0.23 | 0.06 |
| Glycerol g/L | 17.43 | 5.21 | 1.25 |
| Phostphate ppm | 4217 | 3454 | 3353 |
| Ammonia ppm | 2229 | 960 | 882 |
| Description | Feed to Aerobic | Concentration | Concentration |
| | propagator | in Aerobic | in Aerobic |
| | | proagator 1 | proagator 2 |

Ethanol Fermentation

Sugars for fermentation were generated via dilute acid pretreatment, enzymatic hydrolysis, removal of insoluble solids from hydrolyzed feedstock, and concentration of sugars via multiple effect evaporation. To the fermentor, an initial charge of yeast, 1700 gallons, was fed along with 10,300 gallons of switchgrass sugars over 48 hours. The fermentation was allowed to finish from about 48 to about 77 hours. The pH was maintained between about 5.2 to about 5.5 and about temperature at 90° F.

Methanation

Use of the 2 phase acidogenic/methanogenic water treatment system to process centrate resulting from the aerobic propagation of yeast on mixed stillage.

The purpose of the 2 phase methanator is to convert remaining BOD in the yeast centrate to methane and convert sulfate to H₂S, resulting in sour gas. The sour gas may be used by a system to make sulfuric acid.

Energy sorghum cellulosic stillage and defatted concentrated corn stillage was utilized to aerobically propagate GMO yeast for fermentation of Energy sorghum sugar to ethanol as described elsewhere in this patent application at demonstration scale. Following yeast propagation, the yeast was separated by centrifugation generating centrate and yeast paste from aerobic propagation on mixed stillage. The centrate from aerobic propagation on mixed stillage was then fed to a two-phase pilot scale water treatment system for reduction of COD and removal of sulfate. The first phase was operated as a acidogenic reactor at low pH. The effluent from the acidogenic reactor was then fed to a methanogenic reactor. During the course of the two-phase water treatment of centrate the chemical oxygen demand (COD) was reduced generating methane. The sulfate (SO₄) concentration was reduced generating hydrogen Sulfide (H₂S). These combined gasses (sour gas) can be separated by well-known separation processes to generate methane for combustion and sulfur compounds for conversion to sulfuric acid (wet sulfuric acid process). The data generated during this trial is presented below.

Table of Two-Phase Methanation of Yeast
Centrate from Energy Sorghum Feedstock
Feed

| | |
|---|---|
| COD feed mg/L | 8130 |
| sulfate feed ug/ml | 1110 |

Acidogenic Reactor

| | |
|---|---|
| COD effluent mg/L | 3199 |
| COD reduction % | 59 |
| sulfate effluent ug/ml | 501 |
| sulfate reduction % | 53 |

Methanogenic Reactor

| | |
|---|---|
| COD effluent mg/L | 2053 |
| COD reduction % | 74 |
| sulfate effluent ug/ml | 120 |
| sulfate reduction % | 89 |

Single Cell Protein

Following the completion of fermentation to ethanol from switchgrass sugars fermented with GMO yeast as described above the beer had the ethanol removed by distillation. After removal of ethanol, the broth is designated as cellulosic whole stillage. The cellulosic whole stillage was then centrifuged through a disk stack centrifuge to remove insoluble solids. The insoluble solids were then allowed to autolyze (e.g., yeast cell rupturing naturally) or enzymatically hydrolyzed at 120-150° F. in a tank for about 12 to about 24 hours. After autolysis or enzymatic hydrolysis, the cell paste was evaporated through a multiple effect evaporator to 30-40% w/w solids. The resulting concentrate was then spray dried to generate a single cell protein powder with the compositional analysis shown below.

Table of Composition of Single Cell
Protein Generated from Switchgrass

| | Switchgrass | | | |
|---|---|---|---|---|
| Component | average | Stdev | Count | BGY 35 |
| Cysteine % w/w DMB | 0.42 | 0.06 | 17 | 0.58 |
| methionine % w/w DMB | 0.44 | 0.03 | 17 | 0.62 |
| Tryptophan % w/w DMB | 0.23 | 0.04 | 17 | 0.38 |
| Alanine % w/w DMB | 1.76 | 0.23 | 17 | not |
| | | | | reported |
| Arginine % w/w DMB | 0.81 | 0.09 | 17 | 1.83 |
| Aspartic acid % w/w DMB | 1.98 | 0.18 | 17 | not |
| | | | | reported |
| Glutamic acid % w/w DMB | 3.35 | 0.30 | 17 | not |
| | | | | reported |
| Glycine % w/w DMB | 1.16 | 0.08 | 17 | not |
| | | | | reported |
| Histidine % w/w DMB | 0.63 | 0.07 | 17 | 0.85 |
| Isoleucine % w/w DMB | 0.96 | 0.07 | 17 | 1.45 |
| Leucine % w/w DMB | 2.40 | 0.18 | 17 | 3.46 |
| Lysine % w/w DMB | 0.66 | 0.17 | 17 | 1.63 |
| Phenylalanine % w/w DMB | 1.22 | 0.11 | 17 | 2.03 |
| Proline % w/w DMB | 1.28 | 0.11 | 17 | not |
| | | | | reported |
| Serine % w/w DMB | 1.26 | 0.10 | 17 | not |
| | | | | reported |
| Threonine % w/w DMB | 1.19 | 0.08 | 17 | 1.37 |
| Tyrosine % w/w DMB | 0.73 | 0.07 | 17 | not |
| | | | | reported |
| Valine % w/w DMB | 1.30 | 0.10 | 17 | 2.05 |
| ash % w/w DMB | 11.43 | 3.48 | 19 | — |
| Fat content % w/w DMB | 3.49 | 1.62 | 14 | not less |
| | | | | than 5 |
| Protein % w/w DMB | 38.46 | 1.49 | 23 | not less |
| | | | | than 35 |
| Moisture content (% w/w) | 4.71 | 1.26 | 23 | 0 |

Although the subject matter has been described in language specific to structural features and/or methodological acts, it is to be understood that the subject matter defined in the appended claims is not necessarily limited to the specific features or acts described. Rather, the specific features and acts are disclosed as example forms of implementing the claims.

1. A method of producing cellulosic biofuel, the method comprising:
washing feedstock;
pretreating the washed feedstock by adding acid and by adding base for neutralization;
hydrolyzing the pretreated feedstock by adding a cellulase enzyme to produce hydrolysate;
removing suspended solids from the hydrolysate to produce clarified sugars and lignin;
concentrating the clarified sugars to concentrated sugars;
fermenting the concentrated sugars to produce cellulosic biofuel.

2. The method of claim 1, further comprising co-locating a starch ethanol plant to help reduce costs for water and electricity.

3. The method of claim 1, wherein the washing comprises counter-flow washing of the feedstock.

4. The method of claim 1, wherein the washing comprises at least one of a rotary press, a paddle screen, or a washing table.

5. The method of claim 1, wherein the washing the feedstock comprises using water from a scrubber (CO2), from evaporated condensate, or methanator effluent.

6. The method of claim 1, wherein the washing the feedstock improves cellulosic biofuel yield by approximately 1% to approximately 30%.

7. The method of claim 1, further comprising:
generating a slurry from the washed feedstock with evaporator condensate received from the concentrated sugars, wherein the slurry ranges from approximately 1% to approximately 25% in solids;
pumping the slurry to a predetermined pressure; and
injecting steam to the slurry to reach a predetermined temperature;
wherein adding the acid occurs after the slurry has obtained the predetermined pressure.

8. The method of claim 7, further comprising slurrying the washed feedstock with methanator effluent or condensate from salt purge evaporators.

9. The method of claim 7, wherein the predetermined pressure ranges from approximately 0 psig to approximately 300 psig.

10. The method of claim 7, wherein the predetermined temperature ranges from approximately 212° F. to approximately 422° F.

11. A method for pretreatment of the cellulosic feedstock, the method comprising:
using evaporator condensate from concentrated sugars as water source to cellulosic feedstock to create low-solids slurry;
injecting sulfuric acid into the low-solids slurry after it has attained pretreatment pressure; and
adding heat to the low-solids pressurized slurry.

12. The method of claim 11, wherein the heat ranges from approximately 220° F. to approximately 415° F.

13. A method comprising:
combining a cellulosic stillage process stream and a defatted stillage stream into a tank;
adding a base to the tank to create a mixture;
sending the mixture to be combined with a fermenting yeast in a propagation tank to create culture medium with yeast; and
mechanically separating the culture medium combined with yeast to produce yeast paste and/or yeast centrate.

14. The method of claim 13, wherein the defatted stillage stream is from a starch ethanol plant.

15. The method of claim 13, wherein the cellulosic stillage process stream is after distillation in the cellulosic process.

16. The method of claim 13, where the propagation tank is an aerobic process.

17. The method of claim 13, further comprising adding carbon source, aeration, and nutrients for the fermenting yeast.

18. The method of claim 13, wherein an amount of cellulosic stillage process stream ranges from approximately 20% to approximately 60% and an amount of the defatted stillage stream ranges from approximately 40% to approximately 80%.

19. The method of claim 13, wherein an amount of cellulosic stillage process stream ranges from approximately 30% to approximately 70% and an amount of the defatted stillage stream ranges from approximately 30% to approximately 70%.

20. The method of claim 13, wherein the fermenting yeast comprises at least one of a genetically modified organism (GMO), a C5/C6 GMO yeast, a GMO Saccharomyces cerevisiae yeast, a Saccharomyces cerevisiae yeast, an aerobic glycerol consuming yeast, and an ethanol fermenting yeast.

21. The method of claim 1, wherein the fermenting the concentrated sugars comprises use of stillage grown yeast paste.

22. The method of claim 1, wherein the washing comprises use of water at between 71° C. to 106° C., for a duration of 1 to 60 minutes.

23. The method of claim 1, further comprising sending used washwater from the washing step for treatment to toxins, followed by sending the used washwater to a starch ethanol plant for use as slurry make up water and/or as cook water.

24. The method of claim 1, wherein the pretreating comprises adding acid after the feedstock has entered a pressurized zone.

25. The method of claim 1, wherein the feedstock comprises a slurry comprising Energy Sorghum Wash Water and thin stillage.

26. The method of claim 25, where the slurry comprises 0-60% Energy Sorghum Wash Water, thin stillage and the balance water.

27. The method of claim 25, where the slurry further comprises endosperm.

28. The method of claim 1, wherein the step of washing the feedstock comprises washing under conditions that remove one or more of minerals, soluble sugars, potassium, or aflatoxin from the feedstock.

29. The method of claim 1, wherein the step of adding the acid comprises reacting the acid with the washed feedstock for 5 to 10 minutes.
